(19) United States
(12) Patent Application Publication
(10) Pub. No.: US 2011/0039943 A1
(43) Pub. Date: Feb. 17, 2011

(54) METHODS FOR TREATING SKIN DISORDERS WITH TOPICAL NITROGEN MUSTARD COMPOSITIONS

(76) Inventors: Robert Alonso, Rye, NH (US); Martin Stogniew, Blue Bell, PA (US)

Correspondence Address: WILSON, SONSINI, GOODRICH & ROSATI, 650 PAGE MILL ROAD, PALO ALTO, CA, 94304-1050, US

(21) Appl. No.: 12/890,183

(22) Filed: Sep. 24, 2010

Related U.S. Application Data
(63) Continuation-in-part of application No. 12/687,605, filed on Jan. 14, 2010.
Application No. 12/687,605 is a continuation-in-part of application No. 11/369,305, filed on Mar. 7, 2006.
Application No. 12/687,605 is a continuation-in-part of application No. 12/401,812, filed on Mar. 11, 2009.
(60) Provisional application No. 60/661,356, filed on Mar. 14, 2005.
(60) Provisional application No. 60/751,128, filed on Dec. 16, 2005.
(60) Provisional application No. 61/039,840, filed on Mar. 27, 2008.

(51) Int. Cl. A61K 31/131; A61P 17/00; A61P 31/10

(52) U.S. Cl. 514/672

(57) ABSTRACT

Provided are methods for treating skin disorders comprising topically applying to the affected skin a composition comprising a nitrogen mustard or a pharmaceutically acceptable salt thereof.

### CROSS-REFERENCE TO RELATED APPLICATIONS

This application is a continuation-in-part of U.S. application Ser. No. 12/687,605, filed on Jan. 14, 2010, which is a continuation-in-part of U.S. application Ser. No. 11/369,305, filed on Mar. 7, 2006, which claims the benefit of priority to U.S. provisional application Ser. Nos. 60/661,356, filed on Mar. 14, 2005 and 60/751,128, filed on Dec. 16, 2005, the contents of each of which are herein incorporated by reference. U.S. application Ser. No. 12/687,605 is also a continuation-in-part of U.S. application Ser. No. 12/401,812, filed on Mar. 11, 2009, which claims the benefit of priority to U.S. provisional application Ser. No. 61/039,840, filed on Mar. 27, 2008, the contents of each of which are herein incorporated by reference.

### FIELD OF THE INVENTION

The invention encompasses methods for treating skin disorders comprising topically applying to the affected skin a composition comprising a nitrogen mustard or a pharmaceutically acceptable salt thereof.

### BACKGROUND OF THE INVENTION

Alkylating agents, such as nitrogen mustards, have been used in the pharmaceutical industry as anti-cancer drugs. For example, nitrogen mustards have been used to treat cutaneous T-cell lymphoma (CTCL), including mycosis fungoides (MF).

CTCL is a cancer of the white blood cells that primarily affects the skin and only secondarily affects other sites. The disease involves the uncontrolled proliferation of T-lymphocytes known as T-helper (CD4+) cells of the immune system. The proliferation of T-helper cells results in the penetration, or infiltration, of these abnormal cells into the epidermal layer of the skin. The skin reacts with slightly scaling lesions that itch, although the sites of greatest infiltration do not necessarily correspond to the sites of the lesions. The lesions are most often located on the trunk, but can be present on any part of the body. In the most common course of the disease, the patchy lesions progress to palpable plaques that are deeper red and have more defined edges. As the disease worsens, skin tumors develop that are often mushroom-shaped, hence the name mycosis fungoides. Finally, the cancer progresses to extracutanous involvement, often in the lymph nodes or the viscera.

CTCL is a rare disease, with an annual incidence of about 0.29 cases per 100,000 persons in the United States. It is about half as common in Eastern Europe. However, this discrepancy may be attributed to a differing physician awareness of the disease rather than a true difference in occurrence. In the United States, there are about 500-600 new cases a year and about 100-200 deaths. CTCL is usually seen in older adults; the median age at diagnosis is 55-60 years. It strikes twice as many men as women. The average life expectancy at diagnosis is 7-10 years, even without treatment.

Alkylating agents, such as nitrogen mustards, are believed to have anti-cancer activity by acting on the nucleic acids of DNA and RNA. Alkylating agents have four main actions on nucleic acids. First, the agents may cause crosslinking of DNA strands which interferes with DNA and RNA synthesis. This is thought to be the most important reason for the cytotoxic effect of alkylating agents. Secondly, the agents may alter the “side chain groups” of the nucleotide base ring which would lead to abnormal base pairing and point mutations in the synthesized DNA and RNA chains. Thirdly, the alkylating agents may split the base ring from the nucleotide which again interrupts proper DNA and RNA synthesis. Finally, the alkylating agents may break the ring structure of a nucleotide base which would prevent base pairing during DNA and RNA synthesis.

Nitrogen mustards are believed to act as anti-cancer agents by impairing natural DNA strand replication of cancer cells. In natural DNA strand replication, a DNA strand having deoxyribonucleosides, wherein each deoxyribonucleoside may include a base adenine (A), thymine (T), cytosine (C) and guanine (G), replicates by linking each deoxyribonucleoside on the strand with another deoxyribonucleoside, wherein typical linking occurs between adenine (A) and thymine (T), forming an A-T linkage, and between cytosine (C) and guanine (G), forming a C-G linkage, between the original DNA strand and its replicated DNA strand. Nitrogen mustards are believed to allow unnatural base-base linkages such as a guanine (G) base linking to another guanine (G) base if the particular nitrogen mustard is a bifunctional alkylator. As used herein, unless otherwise defined, a bifunctional alkylator is a nitrogen mustard that has at least two 2-chloroethyl side chains, for example, bis-(2-chloroethyl)methyl amine.

Nitrogen mustards may allow unnatural base-base linkages in DNA, for example, by the mechanism depicted in Reactions 1 to 4 below.

First, a nitrogen mustard, such as bis-(2-chloroethyl)methylamine (I) undergoes an intramolecular cyclization, resulting in formation of a highly reactive ethyleniminium intermediate (i.e., a aziridinium cation) (II) according to the following Reaction 1.

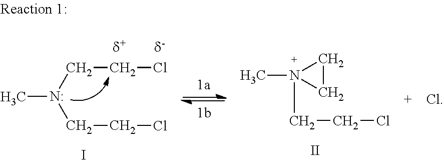

In the bis-(2-chloroethyl)methylamine (I), a carbon atom bonded to chlorine may initially have a partial positive charge, δ+, and a chlorine atom may initially have a partial negative charge, δ−. In Reaction 1, an unshared pair of electrons of nitrogen may form a covalent bond to the carbon having δ+, releasing the chlorine atom as chloride, and forming the ethyleniminium intermediate (II). A concentration of the ethyleniminium intermediate (II) may be in equilibrium with a concentration of the bis-(2-chloroethyl)methylamine (I) wherein the equilibrium constant K_{eq(1a,1b)} may be represented by a ratio of a rate _{k1a,} of the forward reaction 1a, to a rate _{k1b,} of the reverse reaction 1b.

Second, the ethyleniminium intermediate (II) formed in Reaction 1 undergoes nucleophilic attack by an electron donor (i.e., a nucleophile, such as the guanine (III) of DNA), whereby the nucleophile is alkylated to form alkylated deoxyribonucleoside (IV) according to the following Reaction 2.

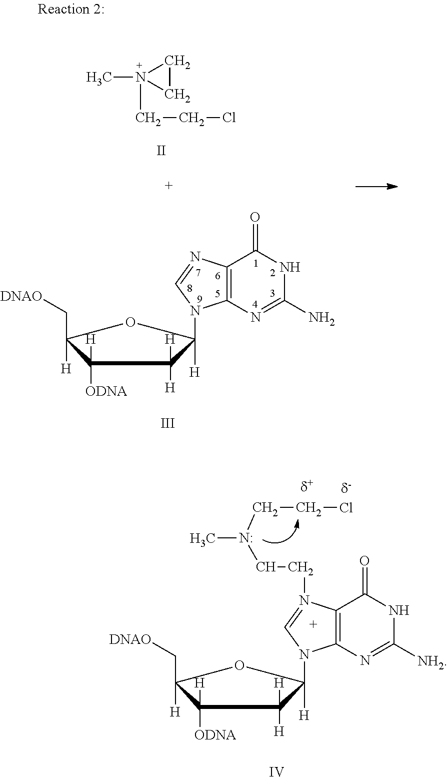

Reaction with the nucleophile guanine (III) at the position N-7 of the guanine occurs to the greatest extent. Other sites on guanine, and other DNA bases, such as adenine, cytosine, and thymine, and phosphate oxygens can also be alkylated.

Third, the alkylchloroethyl side chain of the alkylated deoxyribonucleoside (IV) formed in Reaction 2 undergoes intramolecular cyclization, resulting in formation of deoxyribonuceloside having a highly reactive aziridinium ring (V) according to the following Reaction 3.

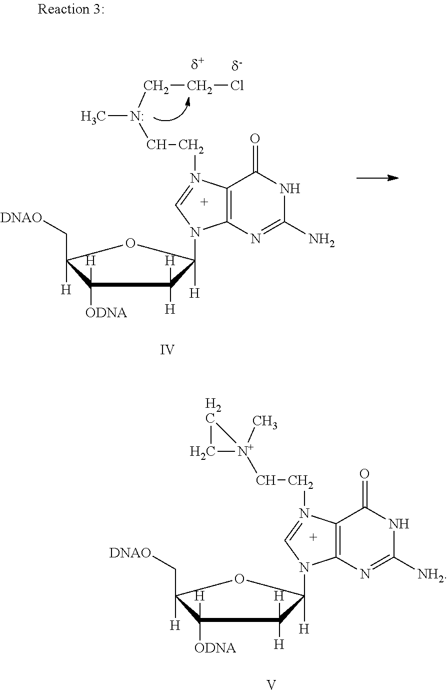

Finally, another guanine (III) of DNA reacts with the deoxyribonucleoside having a highly reactive aziridinium ring (V) formed in Reaction 3 to form an unnatural guanine-guanine link between two strands of DNA, as depicted in Structure (VI), according to the following Reaction 4.

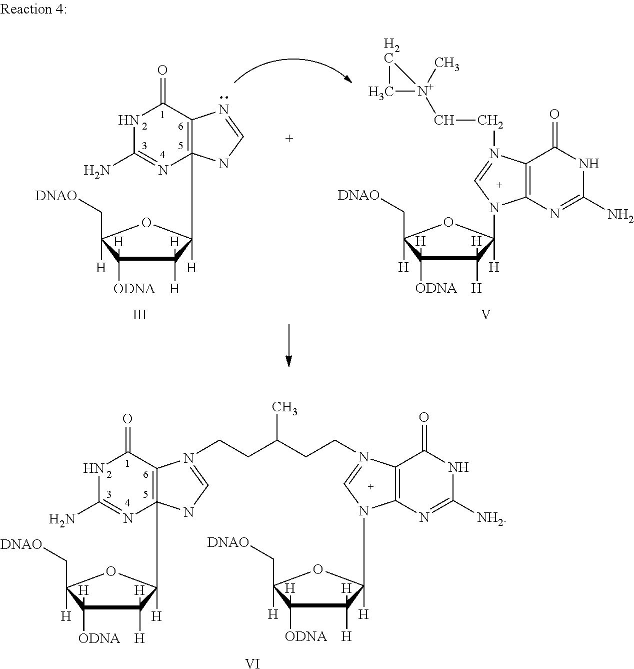

However, only the species which have the potential to form DNA cross-links, i.e. bifunctional species (I and II, Reactions 1-2) are believed to be the active forms of the nitrogen mustard alkylating agents;

The electrophilicity of alkylating agents, such as nitrogen mustards, causes them to be subject to decomposition in the presence of natural nucleophiles in the environment, such as water. In this work HPLC has been employed to determine the degradation products of mechlorethamine in ointment. The degradation (hydrolysis) of mechlorethamine is well characterized giving rise to N-methyl ethanolamine. Alternatively, the drug can degrade by reacting with a wide variety of nucleophiles to form covalent adducts.

Thus, there is a need in the art for stable compositions of alkylating agents, such as nitrogen mustards, that are suitable for topical use.

### SUMMARY OF THE INVENTION

In one embodiment, the invention encompasses methods for treating a skin disorder comprising topically applying to the affected skin a composition comprising an effective amount of an alkylating agent or a pharmaceutically acceptable salt thereof and a pharmaceutically acceptable excipient, wherein the response rate in a group of human patients is greater than about 60% after at least six months of treatment.

In another embodiment, the invention encompasses methods for treating a skin disorder comprising topically applying to the affected skin a composition comprising an effective amount of an alkylating agent or a pharmaceutically acceptable salt thereof and a pharmaceutically acceptable excipient, wherein the response rate in an intent-to-treat group of human patients is greater than about 50%.

In another embodiment, the invention encompasses methods for treating a skin disorder comprising topically applying to the affected skin a composition comprising an effective amount of an alkylating agent or a pharmaceutically acceptable salt thereof and a pharmaceutically acceptable excipient, wherein the response rate in a group of human patients is greater than about 55% upon two months of treatment.

In another embodiment, the invention encompasses methods for treating a skin disorder comprising topically applying to the affected skin a composition comprising an effective amount of bis-(2-chloroethyl)methylamine or a pharmaceutically acceptable salt thereof and a pharmaceutically acceptable excipient, wherein the time to achieve a response rate of 50% in a group of human patients is about 30 weeks or less.

### BRIEF DESCRIPTION OF THE DRAWINGS

FIG. 1 illustrates a method for the use of compositions having stabilized alkylating agents for treating skin disease, in accordance with embodiments of the present invention;

FIGS. 2-4 illustrate a front cross-sectional view of an apparatus having a first compartment, in accordance with embodiments of the present invention;

FIG. 5 depicts a Log-linear plot of the stability of mechlorethamine hydrochloride (MCHCl) in 2-(2-ethoxyethoxy)ethanol at various temperatures over time, in accordance with embodiments of the present invention; and

FIG. 6 depicts rate of MCHCl decomposition vs pH, in accordance with embodiments of the present invention.

FIG. 7 depicts the results of stability testing on MCHCl 0.02% ointment batches at 25° C., as described in Example 8.

FIG. 8 depicts the results of stability testing on MCHCl 0.02% ointment batches at 2-8° C., as described in Example 9.

FIG. 9 depicts the results of stability testing on MCHCl 0.04% ointment batches at 2-8° C., as described in Example 10.

FIG. 10 depicts the results of mass spectrometry analysis measuring the presence and amount of (DP-15) in MCHCl 0.02% ointment batches stored at 2-8° C., as described in Example 11.

FIG. 11 depicts the results of mass spectrometry analysis measuring the presence and amount of (DP-16) in MCHCl 0.02% ointment batches stored at 2-8° C., as described in Example 11.

FIG. 12 illustrates time to response for intent-to-treat patients (as determined by CAILS) when administered mechlorethamine hydrochloride over a period of 12 months according to Example 13.

FIG. 13 illustrates a comparison of primary endpoints for intent-to-treat and efficacy-evaluable patients according to Example 13.

### DETAILED DESCRIPTION OF THE INVENTION

The invention meets a need in the art by providing stable compositions of alkylating agents, such as nitrogen mustards, that are suitable for topical use, and methods of treatment therewith.

Not to be limited by theory, it is believed that nitrogen mustards are highly unstable and have an extremely short duration of action of in the presence of water because water decomposes the highly reactive ethyleniminium intermediate (aziridinium cation), represented by the structure (II) in Reaction 1, supra, replacing one or both chlorine atoms on the 2-chloroethyl side chains of the nitrogen mustard with an OH group.

### I. DEFINITIONS

As used herein, unless otherwise defined, the term “stable,” when referring to a composition of an alkylating agent, means that at least about 80% of the alkylating agent is present in the composition (in other words less than about 20% of the alkylating agent has degraded) after storage. Alternatively, the term “stable” means that the composition contains less than about 20% by weight of degradation product of the alkylating agent after storage.

As used herein, unless otherwise defined, the term “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance, and bioavailability.

As used herein, unless otherwise defined, a “nitrogen mustard prodrug” is a compound that can be metabolized in vivo (i.e., can undergo chemical conversion my metabolic processes) to generate the nitrogen mustard.

As used herein, unless otherwise defined, “topical administration” means applying a drug to a localized area of the body or to the surface of a body part.

As used herein, unless otherwise defined, the term “effective amount” when referring to an alkylating agent means an amount of alkylating agent that is effective to treat a skin disorder.

As used herein, unless otherwise defined, the term “ameliorate” when referring to skin irritation means to lessen pain and reduce skin irritation.

As used herein, unless otherwise defined, the term “room temperature” means a temperature within the range of 15° C. to 30° C.

As used herein, unless otherwise defined, the term “degradation product,” when referring to an alkylating agent, means a compound that can be formed by the degradation of the alkylating agent, for example, by reaction of the alkylating agent with a nucleophile to displace one or more of the functional groups of the alkylating agent.

As used herein, unless otherwise defined, the term “nitrogen mustard degradation product,” means a compound that can be formed by the degradation of a nitrogen mustard, for example, by reaction of the nitrogen mustard with a nucleophile to displace one or more of the terminal chlorides of the nitrogen mustard.

As used herein, unless otherwise defined, the term “response,” when used in connection with treatment of a skin disorder in a human patient, means that the human patient's CAILS after treatment is greater than or equal to 50% lower than the human patient's CAILS prior to treatment and/or the human patient's SWAT score after treatment is greater than or equal to 50% lower than the SWAT score prior to treatment.

As used herein, unless otherwise defined, the term “efficacy-evaluable” or (“EE”), when referring to a patient population enrolled in a drug study, means patients who have received the drug for a period of at least about six months.

As used herein, unless otherwise defined, the term “technically-evaluable” or (“TE”), when referring to a patient population enrolled in a drug study, means all patients who have received at least about two months.

As used herein, unless otherwise defined, the term “intent-to-treat” or (“ITT”), when referring to a patient population enrolled in a drug study, means all patients who have received at least one dose of the drug.

### II. COMPOSITIONS OF ALKYLATING AGENTS

In one embodiment, the invention encompasses a stable composition comprising an alkylating agent or a pharmaceutically acceptable salt or solvate thereof, and at least one pharmaceutically acceptable excipient.

In one embodiment, the composition is in the form of a paste, a dispersion, a suspension, a solution, a gel, a cream, or an ointment. In another embodiment, the composition is in the form of a dispersion. In another embodiment, the dispersion is a coarse dispersion, a colloidal dispersion, or a molecular dispersion.

Suitable alkylating agents include a nitrogen mustard, a sulfur mustard, a Lewisite, an alkyl sulfonate, an ethyleneimine, a nitrosourea, a triazene, an imidazotetrazine, mechlorethamine, chlorambucil, cyclophosphamide, 4-hydroxycyclophosphamide, aldophosphamide, ifosfamide, melphalan, bis-(2-chloroethyl)ethylamine, tris-(2-chloroethyl) ethylamine, carmustine, fotemustine, lomustine, streptozocin, busulfan, dacarbazine, procarbazine, temozolomide, treosulfan, uramustine, hexamethylmelamine, thiotepa (N,N′,N″-triethylenethiophosphoramide), tepa (N,N′, N″-triethylenephosphoramide), and pharmaceutically acceptable salts, solvates, and prodrugs thereof. In one embodiment, the alkylating agent is present in an amount of about 0.001% to about 50% w/w of the composition. In another embodiment, the alkylating agent is present in an amount of about 0.01% to about 0.04% w/w of the composition.

In one embodiment, the alkylating agent is a nitrogen mustard. In one embodiment, the nitrogen mustard is a compound of the following Structure (VII), (VIII), (IX), (X), (XII), (XIII), (XIV), (XV), (XVI), (XVII), (XVIII), or (XIX):

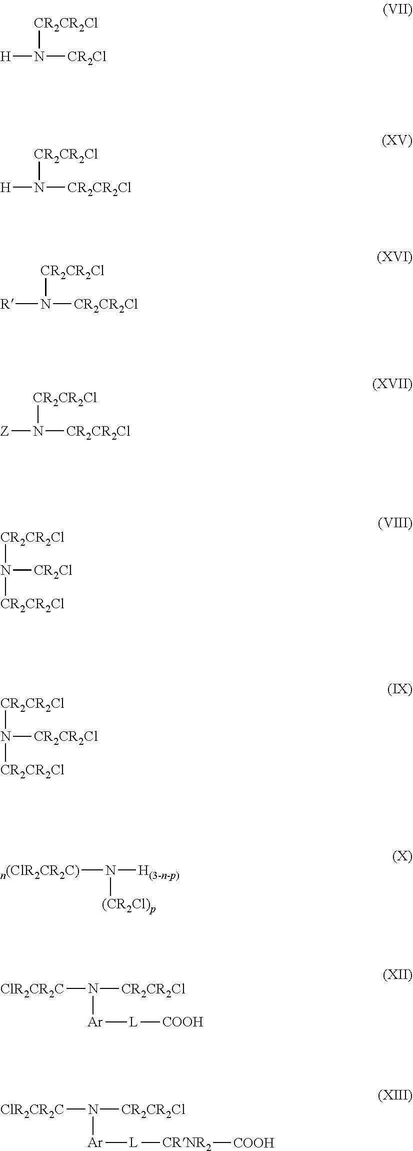

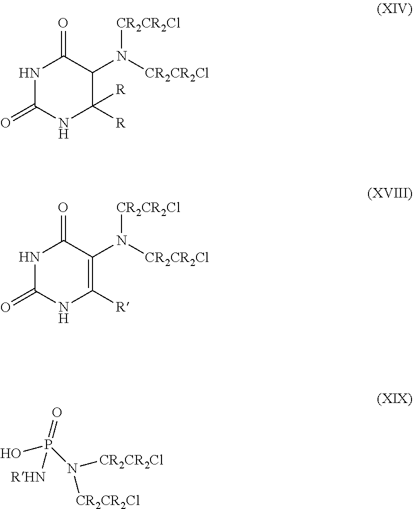

wherein:

each R and R′ is independently selected from the group consisting of H, a linear alkyl group having 1-6 carbon atoms, a branched alkyl group having 2-12 carbon atoms, a cycloalkyl group having 3-17 carbon atoms, a fluorinated linear alkyl group having 2-12 carbon atoms, a fluorinated branched alkyl group having 2-12 carbon atoms, a fluorinated cycloalkyl group having 3-17 carbon atoms, an aryl group, an aralkyl group, an alkaryl group, a cycloalkyl group, a bicycloalkyl group, an alkenyl group, an alkalkenyl group, an alkenylalkyl group, an alkynyl group, an alkalkynyl group, an alkynylalkyl group, a trifluoropropyl group, a cyanopropyl group, an acryloyl group, an arylacryloyl group, an acryloylaryl group, an alkylacyl group, an arylacyl group, an alkylenylacyl group, and an alkynylacyl group, wherein any two R in the same molecule are optionally linked to form a three- to eight-membered cyclic group;

Z is a linear alkyl group having 1-6 carbon atoms;

each L is independently a linking group selected from the group consisting of linear or branched alkylene having 1 to 7 carbon atoms, cycloalkylene having 3 to 17 carbon atoms, alkylcycloalkylene having 4 to 20 carbon atoms, a cycloalkylalkylene having 4 to 20 carbon atoms, an arylene, having 4 to 30 carbon atoms, an alkylarylene, having 4 to 30 carbon atoms, an arylalkylene, having 4 to 30 carbon atoms, and combinations thereof;

each Ar is independently a bifunctional aromatic linking group wherein each Ar is selected from the group consisting of arylene, substituted arylene and heteroarylene;

n is 1, 2, or 3;

p is 0, 1, or 2; and

n+p≦3.

Hereinafter, Structures (VII), (VIII), (IX), (X), (XII), (XIII), (XIV), (XV), (XVI), (XVII), (XVIII), and (XIX) may represent all racemic forms and stereoisomers wherein said compounds may be capable of optical activity.

In one embodiment, each R in Structure (VII), (VIII), (IX), (X), (XII), (XIII), (XIV), (XV), (XVI), (XVII), (XVIII) or (XIX) is hydrogen.

In one embodiment, the nitrogen mustard is a nitrogen mustard of Structure (XVII). In another embodiment, the nitrogen mustard is a nitrogen mustard of Structure (XVII), wherein the Z in structure (XVII) is methyl or ethyl. In another embodiment, the nitrogen mustard is a nitrogen mustard of Structure (XVII), wherein each R in structure (XVII) is independently a linear alkyl group having 1-6 carbon atoms. In another embodiment, the nitrogen mustard is a nitrogen mustard of Structure (XVII), wherein the Z in structure (XVII) is methyl or ethyl and each R in structure (XVII) is independently a hydrogen or linear alkyl group having 1-6 carbon atoms.

In another embodiment, the nitrogen mustard of structure (XVII) is bis-(2-chloroethyl)ethylamine or bis-(2-chloroethyl)methylamine (also known as mechlorethamine).

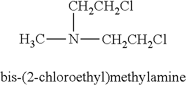

In one embodiment, the nitrogen mustard is a nitrogen mustard of Structure (IX). In another embodiment, the nitrogen mustard of Structure (IX) is tris-(2-chloroethyl)amine.

In one embodiment, the nitrogen mustard is a nitrogen mustard of Structure (XII). In another embodiment, the nitrogen mustard of structure (XII) is chlorambucil of Structure (XIIA):

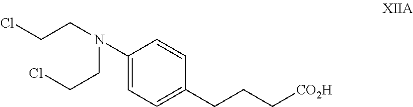

Structure (XII) may be cell cycle-phase nonspecific, although it also may be cytotoxic to nonproliferating cells. Activity may occur as a result of formation of an unstable ethylenimmonium ion, which alkylates or binds with many intracellular molecular structures, including nucleic acids. Its cytotoxic action may be primarily due to cross-linking of strands of DNA, which inhibits nucleic acid synthesis.

In one embodiment, the nitrogen mustard is a nitrogen mustard of Structure (XIII). In another embodiment, the nitrogen mustard of structure (XIII) is melphalan (also known as 4-bis(2-chloroethyl)amino-L-phenylalanine) of Structure (XIIIA):

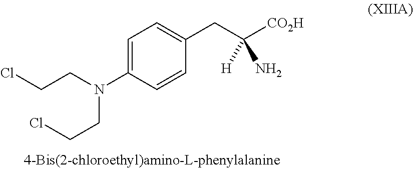

Like the nitrogen mustards of Structure (XII), nitrogen mustards of Structure (XIII) may be cell cycle-phase nonspecific, although they also may be cytotoxic to nonproliferating cells.

In one embodiment, the nitrogen mustard is a nitrogen mustard of Structure (XVIII). In another embodiment, the nitrogen mustard of structure (XVIII) is uracil mustard of Structure (XVIIIA):

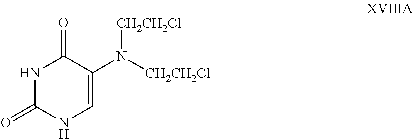

In one embodiment, the nitrogen mustard, pharmaceutically acceptable salt of the nitrogen mustard, or prodrug of the nitrogen mustard is present in an amount of about 0.0001% to about 10% by weight of the composition. In another embodiment, the nitrogen mustard, pharmaceutically acceptable salt of the nitrogen mustard, or prodrug of the nitrogen mustard is present in an amount of about 0.001% to about 2.0% by weight of the composition. In another embodiment, the nitrogen mustard, pharmaceutically acceptable salt of the nitrogen mustard, or prodrug of the nitrogen mustard is present in an amount of 0.01% to about 0.1% by weight of the composition. In another embodiment, the nitrogen mustard, pharmaceutically acceptable salt of the nitrogen mustard, or prodrug of the nitrogen mustard is present in an amount of 0.01% to about 0.04% by weight of the composition. In another embodiment, the nitrogen mustard, pharmaceutically acceptable salt of the nitrogen mustard, or prodrug of the nitrogen mustard is present in an amount of 0.015% to about 0.04% by weight of the composition. In another embodiment, the nitrogen mustard, pharmaceutically acceptable salt of the nitrogen mustard, or prodrug of the nitrogen mustard is present in an amount of 0.015% to about 0.03% by weight of the composition.

In another embodiment, the nitrogen mustard is in the form of a pharmaceutically acceptable salt. Suitable pharmaceutically acceptable salts of nitrogen mustard include HX salts of the following Structures (VIIa), (VIIIa), (IXa), (Xa), (XIIa), (XIIIa), (XIVa), (XVa), (XVIa), (XVIIa), (XVIIIa), and (XIXa):

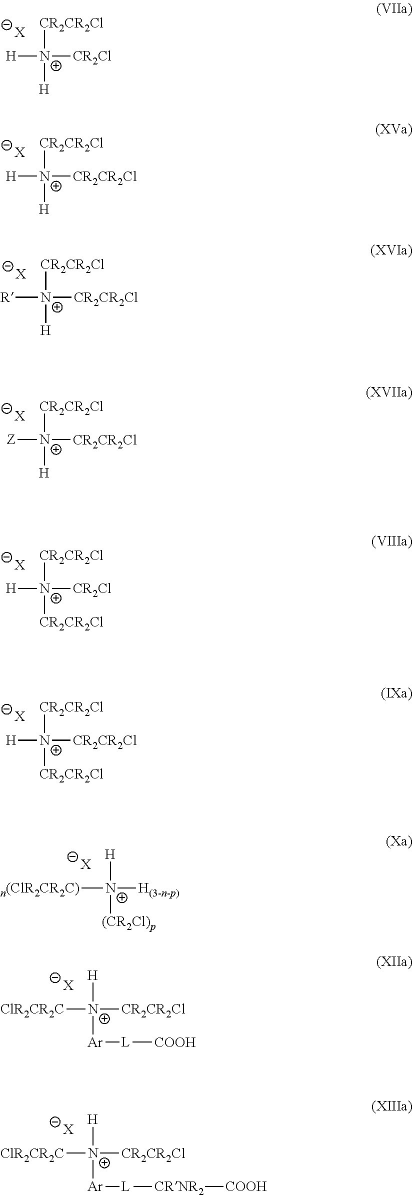

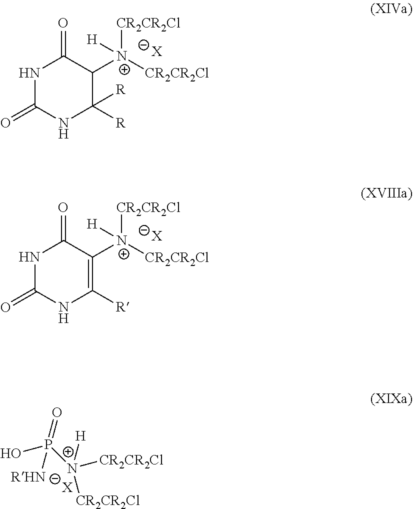

wherein R, R′. Z, Ar, L, n, and p are as defined above for the compounds of Structures (VII), (VIII), (IX), (X), (XII), (XIII), (XIV), (XV), (XVI), (XVIII), and (XIX).

In one embodiment, X⁻ is a halide, such as Cl⁻, Br, or I⁻, or HSO₄⁻ or NO₃⁻. The corresponding HX is HCl, HBr, HI, or H₂SO₄, or HNO₃, respectively. In another embodiment, the pharmaceutically acceptable FIX salt is a conventional acid-addition salt or base-addition salt formed from a non-toxic organic or inorganic acid or inorganic base. Illustrative acid-addition salts include those derived from inorganic acids such as hydrochloric acid, hydrobromic acid, hydroiodic acid, sulfuric acid, sulfamic acid, phosphoric acid, and nitric acid, and those derived from organic acids such as p-toluenesulfonic acid, methanesulfonic acid, ethane-disulfonic acid, isethionic acid, oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, and fumaric acid. Illustrative base-addition salts include those derived from ammonium hydroxides (e.g., a quaternary ammonium hydroxide such as tetramethylammonium hydroxide), those derived from inorganic bases such as alkali or alkaline earth-metal (e.g., sodium, potassium, lithium, calcium, or magnesium) hydroxides, and those derived from non-toxic organic bases such as basic amino acids.

In some embodiments of the invention, use of the pharmaceutically acceptable HX salt of the nitrogen mustard, for example of structure (XXa, infra), may preserve the effective alkylating activity by reducing its volatility compared to that of the free base form of the nitrogen mustard, since pharmaceutically acceptable HX salts of nitrogen mustards generally have lower vapor pressures than their corresponding free base forms.

Reaction 5 illustrates competing equilibrium reactions, 1a and 1b and 5a and 5b. The reaction represented by arrow 5c, infra, illustrates the conversion of a free base nitrogen mustard to a pharmaceutically acceptable HX salt by reaction of the nitrogen mustard with HX.

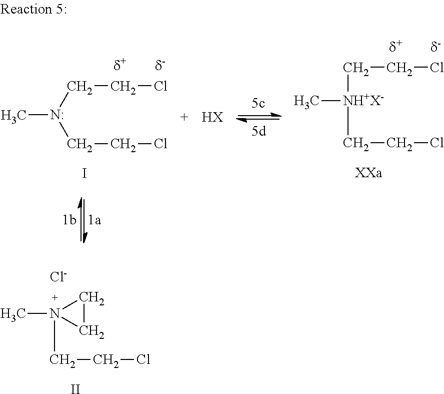

In Reactions 1a and 1b, a free form of the nitrogen mustard, structure (I), may be in equilibrium with the aziridium ion (II), as described for Reaction 1, supra. The equilibrium constant for Reactions 1a and 1b has been described as K_{eq(1a,1b)}, supra. In like manner, the equilibrium constant for Reactions 5a and 5b, K_{eq(5a,5b)} may be expressed as the ratio of the concentration of the HX salt, (XXa), to the product of the concentration of the free base form of the nitrogen mustard, structure (I) and the concentration of HX. Therefore, in one embodiment, there may be an equilibrium concentration of aziridinium cation represented by the ratio of K_{eq(1a,1b)} to K_{eq(5a,5b),} even when the nitrogen mustard has been stabilized by converting the free base form of the nitrogen mustard, as represented by structure (I), infra, as illustrated by Reaction 5, to its HX salt, as represented by the structure (XXa). Therefore, the N-2 position of the guanine base of DNA, structure III in Reactions 2-4 may be alkylated by the HX salt (XXa), as in Reaction 5, because the concentration of the aziridinium cation in Reaction 5, may be a real positive number, equal to K_{eq(1a,1b)} to K_{eq(5a,5b)}. Hereinafter, the free base form of the nitrogen mustard is any non-salt form of the nitrogen mustard, wherein a lone pair of electrons on the nitrogen atom may be available for forming the aziridinium ion, (II), as in Reaction 1, supra. In embodiments of the present invention, the aziridinium cation, Structure (II), supra, may undergo nucleophilic attack by an electron donor, resulting in alkylating the nucleophile. For example, reaction with the nucleophile guanine (G), structure (III), shown in Reaction 2, supra, at position N-7 of the guanine (G) occurs to the greatest extent. Other sites on guanine (G), and other DNA bases such as adenine (A), cytosine (C) and thymine (T), and phosphate oxygens also can be alkylated.

In another embodiment, the nitrogen mustard is provided in the form of a nitrogen mustard prodrug. Suitable nitrogen mustard prodrugs include those of the following Structure (XI):

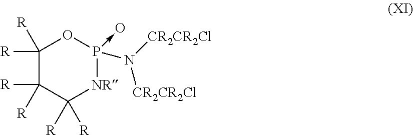

wherein each R and each R″ is independently selected from the group consisting of H, a linear alkyl group having 1-6 carbon atoms, a branched alkyl group having 2-12 carbon atoms, a cycloalkyl group having 3-17 carbon atoms, a fluorinated linear alkyl group having 2-12 carbon atoms, a fluorinated branched alkyl group having 2-12 carbon atoms, a fluorinated cycloalkyl group having 3-17 carbon atoms, an aryl group, an aralkyl group, an alkaryl group, a cycloalkyl group, a bicycloalkyl group, an alkenyl group, an alkalkenyl group, an alkenylalkyl group, an alkynyl group, an alkalkynyl group, an alkynylalkyl group, a trifluoropropyl group, a cyanopropyl group, an acryloyl group, an arylacryloyl group, an acryloylaryl group, an alkylacyl group, an arylacyl group, an alkylenylacyl group, and an alkynylacyl group, wherein any two R in the same molecule are optionally linked to form a three- to eight-membered cyclic group. Hereinafter, Structure (XI) may represent all racemic forms and stereoisomers wherein said compounds may be capable of optical activity.

For example, phosphatase and phosphamidase enzymes may cleave the P—N bond of Structure (XI), supra, e.g., cyclophosphamide, Structure (XIA), infra or ifosphamide, Structure (XIB), infra, resulting in an intermediate aldophosphamide, which may nonenzymatically break down to a bifunctional phosphoramide mustard, for example of Structure (XIXA) or (XIXB), as illustrated in Reactions 6a and 6b below. In an embodiment, cyclophosphamide, Structure (XIA), supra or ifosphamide, Structure (XIB), supra may be oxidatively activated by cytochrome P-450.

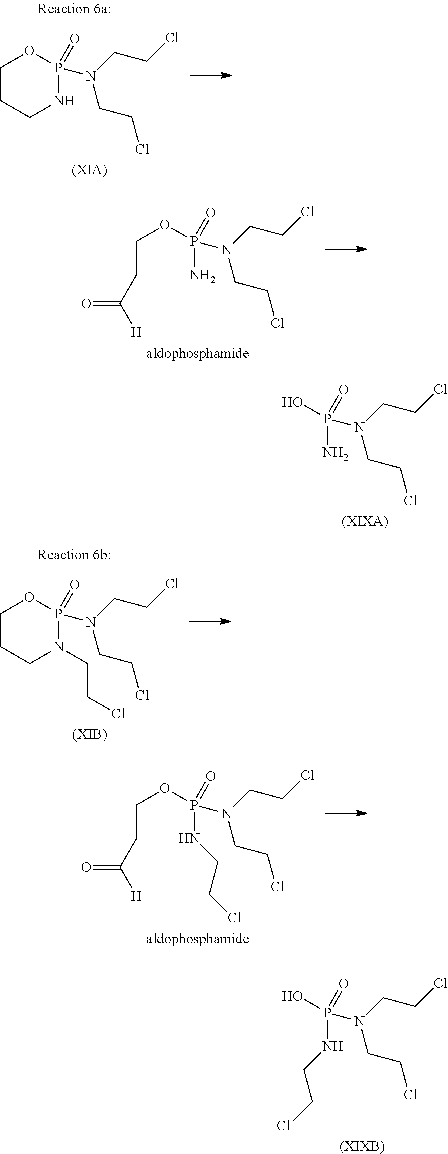

In one embodiment, the pharmaceutically acceptable excipient is a non-aqueous vehicle or carrier. In one embodiment, the non-aqueous vehicle or carrier does not include petrolatum, ethanol, or acetone. In another embodiment, the non-aqueous vehicle or carrier is present in an amount of less than 98% by weight of the composition. In another embodiment, the non-aqueous vehicle or carrier is present in an amount of about 15% to about 60% by weight of the composition.

In one embodiment, the non-aqueous vehicle or carrier comprises an ingredient selected from the group consisting of a secondary alcohol, a tertiary alcohol, an amine, an amino alcohol having 1 to 20 carbon atoms, a polypropylene glycol (PPG), a propylene glycol (PG), a polyethyleneglycol (PEG), a diethylene glycol monosubstituted ether, and a diethylene glycol monomethyl ether (DGME).

In one embodiment, the non-aqueous vehicle or carrier comprises a diethylene glycol monosubstituted ether. In another embodiment, the diethylene glycol monosubstituted ether is a compound of the formula HOCH₂CH₂OCH₂CH₂OR⁷⁹ or (HO(CH₂CH₂O)₂R⁷⁹), wherein R⁷⁹ is selected from the group consisting of a linear alkyl group having 1-6 carbon atoms, a branched alkyl group having 2-12 carbon atoms, a cycloalkyl group having 3-17 carbon atoms, a fluorinated linear alkyl group having 2-12 carbon atoms, a fluorinated branched alkyl group having 2-12 carbon atoms, and a fluorinated cycloalkyl group having 3-17 carbon atoms, an aryl group, an aralkyl group, an alkaryl group, a cycloalkyl group, a bicycloalkyl group, an alkenyl group, an alkalkenyl group, an alkenylalkyl group, an alkynyl group, an alkalkynyl group, an alkynylalkyl group, a trifluoropropyl group, a cyanopropyl group, an acryloyl group, an arylacryloyl group, an acryloylaryl group, an alkylacyl group, an arylacyl group, an alkylenylacyl group and an alkynylacyl group, and combinations thereof. In another embodiment, R⁷⁹ is a linear alkyl group having 1-6 carbon atoms, a branched alkyl group having 2-12 carbon atoms. In another embodiment, R⁷⁹ is a linear alkyl group having 1-6 carbon atoms. In another embodiment, R⁷⁹ is a linear alkyl group having 2 to 3 carbon atoms. In another embodiment, R⁷⁹ is ethyl, which corresponds to ethoxy diglycol reagent (also known as diethylene glycol monoethyl ether, 2-(2-ethoxyethoxy)ethanol or Transcutol®).

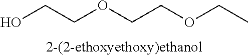

In another embodiment, the non-aqueous vehicle or carrier comprises a polyoxylglyceride. In one embodiment, the polyoxylglyceride is a caprylocaproyl, linoleoyl, oleoyl, lauroyl, or stearoyl polyoxylglyceride. In another embodiment, the polyoxylglyceride is lauroyl polyoxyl-32 glycerides, stearoyl polyoxyl-32 glycerides, medium chain triglycerides, oleoyl polyoxyl-6 glycerides, linoleoyl polyoxyl-6 glycerides, lauroyl polyoxyl-6 glycerides, or caprylocaproyl polyoxyl-8-glycerides. Such polyoxylglycerides are available from Gattefosse (Canada) under the tradenames Labrasol®, Labrafil®, and Gelucire®.

In one embodiment, the PPG has a molecular weight from about 300 to about 2500. In another embodiment, the PEG has a molecular weight from about 100 to about 5000.

In one embodiment, the secondary or tertiary alcohol is isopropyl alcohol, cetyl alcohol, stearyl alcohol, cetearyl alcohol, or lanolin alcohol.

In some embodiments, the nitrogen mustard alkylating agents disclosed herein are bifunctional alkylators, i.e., have two arms terminated with chlorine (“CR₂CR₂Cl”) that can react, for example, with DNA to form DNA cross-links as illustrated in Reactions 1 to 4. When one arm terminated with chlorine is absent, the nitrogen mustard alkylating agent is referred to as a monofunctional alkylator or a “half-mustard.”

It is believed that nucleophiles in the composition or in the environment may degrade the nitrogen mustard alkylating agent to form a nitrogen mustard degradation product by reacting with the nitrogen mustard to displace one or more terminal chlorides of the nitrogen mustard by nucleophilic substitution.

Nucleophiles are defined as molecules having electron-rich functional groups (“E”), such as —O—, —NH—, or —S—. The most nucleophilic nucleophiles are believed to be water or nucleophiles having the electron-rich functional group covalently bonded to a primary carbon atom, such as methanol or ethanol. Nucleophiles include any pharmaceutically acceptable excipient having an electron-rich functional group (E) known to the skilled artisan. Such pharmaceutically acceptable excipients include, but are not limited to, acidifying agents, adsorbants, alkalizing agents, antibacterial agents, antifoaming agents, antiseptics, antiviral agents, binding agents, buffering agents, bulking agents, chelating agents, coating agents, coloring agents, release-modifying agents, cooling agents, diluents, disintegrants, dispersing agents, emollients, emulsifying agents, film-forming agents, gelling agents, glidants, granulating agents, humectants, lubricants, ointment bases, opacifying agents, oleaginous vehicles, penetration enhancers, pH-adjusting agents, pigments, plasticizers, preservatives, refrigerants, sequestering agents, solubilizing agents, solvents, stabilizing agents, stiffening agents, surfactants, suspending agents, sweetening agents, thickening agents, transdermal delivery agents, tonicity agents, and wetting agents. Pharmaceutically acceptable excipients are described, for example, in the HANDBOOK OF PHARMACEUTICAL EXCIPIENTS (5th ed., 2006, R. C. Rowe, et al., eds.), the contents of which are herein incorporated by reference.

The degradation of a nitrogen mustard alkylating agent by a nucleophile having an electron-rich functional group is illustrated, for example, by Reactions 7a and 7b below:

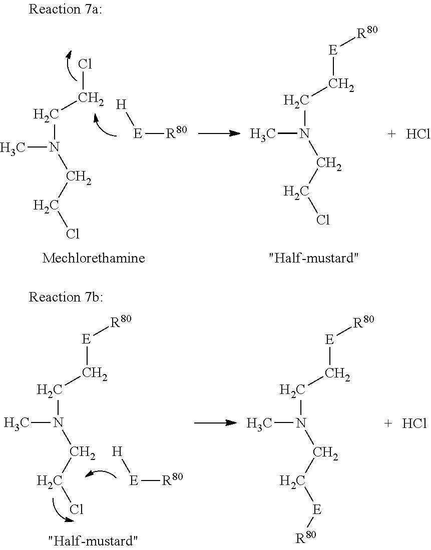

wherein each R80 is independently a linear or branched alkyl group having 1-12 carbon atoms that is optionally substituted with one or more —COOH or —OH, and that is optionally interrupted by one or more —O—, —N—, —(CO)—, or —O—(CO)—. As used herein, the term “interrupted,” when referring to an alkyl group, means that one or more of the carbon-carbon bonds of the alkyl group is replaced with a —O—, —N—, —(CO)—, or —O—(CO)—, for example, as follows:

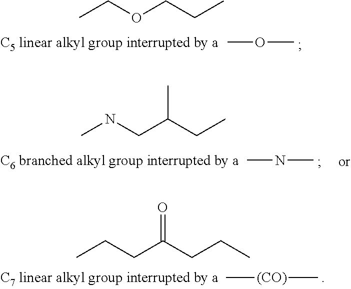

It has surprisingly been found by the present inventors that such degradation reactions may be reduced and/or avoided by the presence of PEG, EG, PPG, or PG, or diethylene glycol monosubstituted ethers, such as 2-(2-ethoxyethoxy)ethanol, in the composition. Not to be limited by theory, it is believed that these excipients hydrogen bond to nucleophiles that may be present in the composition or the environment, thereby reducing the nucleophilic strength of the nucleophiles and reducing their ability to degrade the nitrogen mustard.

In another embodiment, the composition further comprises an adjuvant. Suitable adjuvants include, but are not limited to, antioxidants, preserving agents, stabilizing agents, wetting agents, emulsifying agents and the like. In other embodiments, the composition further comprises a solvent, an antioxidant, an emollient, a humectant, a preservative, an emulsifier, a pH agent, or a combination thereof.

Suitable solvents include acetone, glycols, polyurethanes, and others known in the art. Suitable emollients include mineral oil, propylene glycol dicaprylate, lower fatty acid esters, lower alkyl ethers of propylene glycol, cetyl alcohol, cetostearyl alcohol, stearyl alcohol, stearic acid, wax, and others known in the art.

Suitable antioxidants include sodium bisulfite, butylated hydroxytoluene, edetate disodium, benzyl alcohol, ascorbic acid, citric acid, malic acid, fumaric acid, lactic acid, and propionic acid, and mixtures thereof. In one embodiment, the antioxidant is sodium bisulfite, butylated hydroxytoluene, or edetate disodium, or a mixture thereof.

Suitable humectants include glycerin, sorbitol, and others known in the art.

Suitable emulsifiers include glyceryl monostearate, glyceryl monoleate, stearic acid, polyoxyethylene cetyl ether, polyoxyethylene cetostearyl ether, polyoxyethylene stearyl ether, polyethylene glycol stearate, propylene glycol stearate, and others known in the art.

Suitable pH agents include hydrochloric acid, phosphoric acid, diethanolamine, triethanolamine, sodium hydroxide, monobasic sodium phosphate, dibasic sodium phosphate, and others known in the art. Suitable pH agents also include organic acids, for example, of the formula CₙH₍₂ₙ₊₂₎COOH, (where n is an integer of 1 to 6). Suitable organic acids include, but are not limited to, acetic acid, citric acid, tartaric acid, fumaric acid, lactic, glycolic and other alpha hydroxy acids, malic acid, carnitine, glutamic acid, aspartic acid and others known in the art. In one embodiment, the organic acid is present in am amount of about 0.01 percent to about 15 percent by weight of the composition. In another embodiment, the organic acid is present in an amount of about 1 percent to about 15 percent by weight of the composition. In another embodiment, the organic acid is present in an amount of about 2 percent to about 5 percent by weight of the composition. In one embodiment, the organic acid is present in the composition in an amount sufficient to provide a pH of less than about 7. In another embodiment, the organic acid is present in the composition in an amount sufficient to provide a pH of less than 5. In another embodiment, the organic acid is present in the composition in an amount sufficient to provide a pH of less than about 4. In another embodiment, the organic acid is present in the composition in an amount sufficient to provide a pH of about 3 to about 4. In another embodiment, the organic acid is present in an amount sufficient to provide a pH of about 2.5 to about 3.5. In another embodiment, the organic acid is present in the composition in an amount sufficient to provide a pH of about 3.

Suitable preservatives include benzyl alcohol, sodium benzoate, parabens, and others known in the art.

In one embodiment, the composition further comprises a dimethyl polysiloxane fluid. In one embodiment, the dimethyl polysiloxane fluid is a dimethicone or cyclodimethicone. In one embodiment, the dimethyl polysiloxane fluid has essentially no moisture content.

As used herein, the term “dimethicone” includes low viscosity silicones, low viscosity, i.e. from about 1 cps to about 1,000 cps at 25° C. polydimethylsiloxanes, Hexamethyldisiloxane (CAS#107-46-0), pure silicone 1 cSt, volatile silicone, volatile silicones, volatile polydimethylsiloxanes, low temperature silicones, skin care silicone, skin care silicones, Octamethyltrisiloxane (CAS#107-51-7), Decamethyltetrasiloxane (CAS#141-62-8), Dodecamethylpentasiloxane (CAS#141-63-9), trisiloxane, low viscosity dimethicone, volatile dimethicone, cosmetic dimethicone fluid, cosmetic base fluids, suntan lotion silicone, antiperspirant silicone, hair care silicone, low surface tension silicone, and low heat of vaporization silicone.

As used herein, the term “cyclomethicone” includes cyclopentasiloxane, volatile poydimethylcyclosiloxane (CAS#541-02-6), low surface tension silicone, volatile silicone, D5 silicone, Dow Corning 245 fluid, DC 245 fluid, 245 silicone, skin cream silicone, antiperspirant silicone, suntan lotion silicone, silicone for skin, skincare silicone, bodycare silicone, bath oil silicone, GE 1202, GE SF1202 cyclopentasiloxane, D5 Cyclopentasiloxane, and D5 Decamethylcyclopentasiloxane.

Generally, dimethicone and cyclomethicone are dimethyl silicone oils with good emollience, strong moisturization and humectant properties. Dimethicone and cyclomethicone have very low moisture content, as water, i.e. <0.1% by weight because they are methyl stopped instead of OH stopped polymers.

In one embodiment of the invention, the composition comprises an alkylating agent or a pharmaceutically acceptable salt thereof, and a pharmaceutically acceptable excipient selected from the group consisting of 2-(2-ethoxyethoxy)ethanol, Hydroxypropylcellulose, Menthol Crystals USP, Butylated Hydroxytoluene NF, Glycerin USP, Edetate Disodium USP, Decyl Methyl Sulfoxide, Kris-Ester 236 and combinations thereof.

In one embodiment, the composition does not include any grade of white or yellow petrolatum recognized in the art as suitable for human application. In another embodiment, the non-aqueous vehicle or carrier does not include material commercially available as Penreco Snow White Pet USP. In another embodiment, the composition does not include hydrocarbon mixtures formulated with mineral oils in combination with paraffin waxes of various melting points. In another embodiment, the composition does not include a lipophilic emollient selected from the group consisting of: petrolatum; and esters of fatty acids.

In some embodiments, the composition does not comprise an inorganic salt. In other embodiments, the composition does not comprise an antioxidant.

In one embodiment, the composition does not comprise water or ethanol. In some embodiments, the composition comprises less than about 15% by weight water, less than about 10% by weight water, less than about 5% by weight water, or less than about 1% by weight water. In other embodiments, the composition comprises less than about 15% by weight ethanol, less than about 10% by weight ethanol, less than about 5% by weight ethanol, or less than about 1% by weight ethanol. In one embodiment, the composition does not comprise petrolatum. In some embodiments, the composition comprises less than about 15% by weight petrolatum, less than about 10% by weight petrolatum, less than about 5% by weight petrolatum, or less than about 1% by weight petrolatum. In some embodiments, the composition comprises less than about 15% by weight acetone, less than about 10% by weight acetone, less than about 5% by weight acetone, or less than about 1% by weight acetone.

In one embodiment, the pH of the composition is less than about 7. In another embodiment, the pH of the composition is less than 5. In another embodiment, the pH of the composition is less than about 4. In another embodiment, the pH of the composition is about 3 to about 4. In another embodiment, the pH of the composition is about 2.5 to about 3.5. In another embodiment, the pH of the composition is about 3.

In one embodiment, the viscosity of the composition is more than the viscosity of water (about 1 cps) and less than the viscosity of petrolatum (about 64,000 cps). In another embodiment, the viscosity of the composition is about 5,000 cps to about 50,000 cps. In another embodiment, the viscosity of the composition is about 15,000 cps to about 40,000 cps. In another embodiment, the viscosity of the composition is about 20,000 cps to about 35,000 cps. In another embodiment, the viscosity of the composition is about 25,000 cps to about 35,000 cps. Viscosity can be measured with a Brookfield programmable rheometer, model RVDV-III with cone plate configuration using spindle CPE52, or equivalent apparatus. Viscosity measurements can be taken at 25° C. and 1 rpm over a period of 5-10 minutes, using a 0.5 mL sample size.

In one embodiment, the composition has a duration of activity from about 3 months to about 3 years.

In one embodiment, the composition is stable, i.e., at least about 80% of the alkylating agent is present in the composition or less than about 20% by weight degradation product of the alkylating agent is present in the composition after storage. In one embodiment, the composition is stored at a temperature of at least about −20° C. In another embodiment, the composition is stored at a temperature of about −20° C. to about −10° C. In one embodiment, the composition is stored at a temperature of at least about 2° C. In another embodiment, the composition is stored at a temperature of about 2° C. to about 8° C. In another embodiment, the composition is stored at room temperature. In another embodiment, the composition is stored at about 25° C. In another embodiment, the composition is stored for about 3 months to about 3 years.

In one embodiment, at least about 80% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 85% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 90% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 95% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 98% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, at least about 99% of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months.

In one embodiment, less than about 20% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 15% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 10% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 5% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 1% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months. In one embodiment, less than about 0.5% by weight degradation product of the alkylating agent is present in the composition after storage for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months.

In one embodiment, the composition is stored in a glass vial sealed from the atmosphere. In another embodiment, the composition is stored in an amber vial sealed from the atmosphere. In another embodiment, the composition is stored in an aluminum foil-lined container. In another embodiment, the composition is stored in an aluminum foil tube.

In another embodiment, the composition is stored in a plastic container. In another embodiment, the composition is stored in a polypropylene container.

In one embodiment, the composition is stable in the presence of water. In another embodiment, the composition is stable and comprises 1%, 2%, 5%, 10%, 15%, or 20% by weight water.

In one embodiment, the alkylating agent is a nitrogen mustard and the degradation product is a nitrogen mustard degradation product.

In some embodiments, the nitrogen mustard degradation product is a half-mustard. In some embodiments, the half-mustard has the following structure (DP-A) or (DP-B):

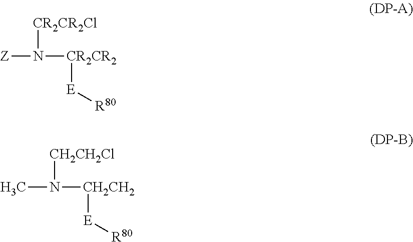

wherein:

Z is a linear alkyl group having 1-6 carbon atoms;

each R is independently hydrogen or a linear alkyl group haying 1-6 carbon atoms;

each E is independently —O—, —NH—, or —S—; and

each R⁸⁰ is independently a linear or branched alkyl group having 1-12 carbon atoms that is optionally substituted with one or more —COOH or —OH, and that is optionally interrupted by one or more —O—, —N—, —(CO)—, or —O—(CO)—.

In some embodiments, the moiety E-R⁸⁰ is

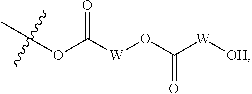

wherein W is a linear or branched alkyl group having 1-6 carbon atoms that is optionally substituted with —COOH. In another embodiment, the moiety E-R⁸⁰ is

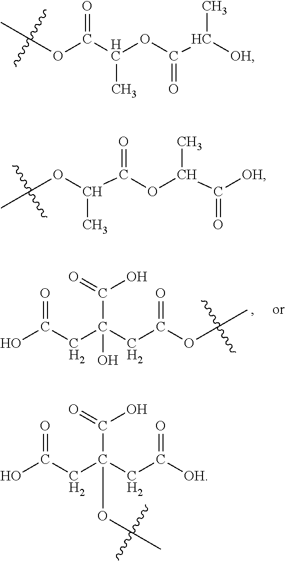

In one embodiment, the moiety E-R⁸⁰ is

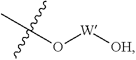

wherein W′ is a linear or branched alkyl group having 1-6 carbon atoms. In another embodiment, the moiety E-R⁸⁰ is

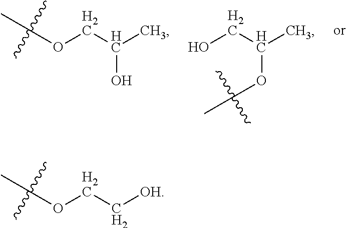

In other embodiments, the half-mustard has the structure (DP-A) or (DP-B), wherein Z is a linear alkyl group having 1-6 carbon atoms; each E is independently —O—, —NH—, —S—; —OC(O)CH(CH₃)OC(O)CH(CH₃)—; —OCH(CH₃)C(O)OCH(CH₃)—; or —O(CH₂)₂O(CH₂)₂O—; and each R⁸⁰ is independently a linear or branched alkyl group having 1-12 carbon atoms, —COOH, or —OH.

In other embodiments, the nitrogen mustard degradation product has the following structure (DP-C) or (DP-D):

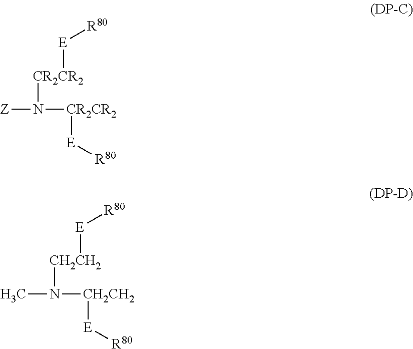

wherein:

Z is a linear alkyl group having 1-6 carbon atoms;

each R is independently hydrogen or a linear alkyl group having 1-6 carbon atoms;

each E is independently —O—, —NH—, or —S—; and

each R⁸⁰ is independently a linear or branched alkyl group having 1-12 carbon atoms that is optionally substituted with one or more —COOH or —OH, and that is optionally interrupted by one or more —O—, —N—, —(CO)—, or —O—(CO)—.

In some embodiments, each E-R⁸⁰ moiety is independently

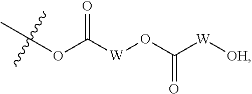

wherein W is a linear or branched alkyl group having 1-6 carbon atoms that is optionally substituted with —COOH. In another embodiment, the each E-R⁸⁰ moiety is independently

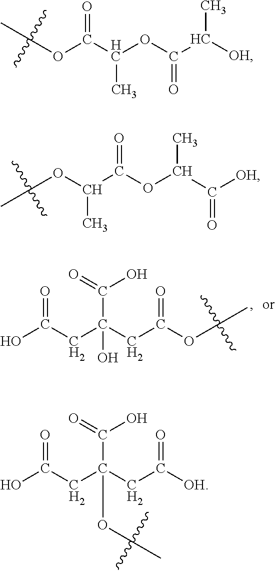

In one embodiment, each E-R⁸⁰ moiety is independently

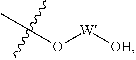

wherein W′ is a linear or branched alkyl group having 1-6 carbon atoms. In another embodiment, each E-R⁸⁰ moiety is independently

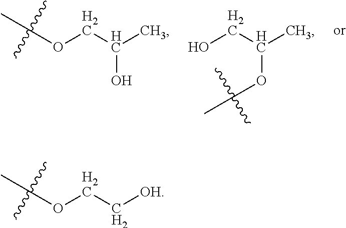

In other embodiments, nitrogen mustard degradation product has the structure (DP-C) or (DP-D), wherein Z is a linear alkyl group having 1-6 carbon atoms; each E is independently —O—, —NH—, —S—; —OC(O)CH(CH₃)OC(O)CH(CH₃)—; —OCH(CH₃)C(O)OCH(CH₃)—; or —O(CH₂)₂O(CH₂)₂O—; and each R⁸⁰ is independently a linear or branched alkyl group having 1-12 carbon atoms, —COOH, or —OH.

### III. METHODS FOR STABILIZING ALKYLATING AGENTS

In one embodiment, the invention provides methods for stabilizing an alkylating agent comprising combining the alkylating agent with a pharmaceutically acceptable excipient to provide a stable composition of the alkylating agent.

In one embodiment, the alkylating agent is a nitrogen mustard. In another embodiment, the alkylating agent is a nitrogen mustard of Structure (VII), (VIII), (IX), (X), (XII), (XIII), (XIV), (XV), (XVI), (XVII), (XVIII) or (XIX). In another embodiment, the nitrogen mustard is bis-(2-chloroethyl)ethylamine, bis-(2-chloroethyl)methylamine, or tris-(2-chloroethyl)amine. In another embodiment, the nitrogen mustard is bis-(2-chloroethyl)methylamine.

In one embodiment, the pharmaceutically acceptable excipient is polypropylene glycol (PPG), propylene glycol (PG), polyethylene glycol (PEG), ethylene glycol (EG), or 2-(2-ethoxyethoxy)ethanol. In another embodiment, the pharmaceutically acceptable excipient is 2-(2-ethoxyethoxy)ethanol. In another embodiment, the pharmaceutically acceptable excipient consists essentially of Propylene Glycol, 2-(2-ethoxyethoxy)ethanol, Hydroxypropylcellulose, Menthol Crystals USP, Butylated Hydroxytoluene NF, Glycerin USP, Edetate Disodium USP, Decyl Methyl Sulfoxide, and Kris-Ester 236. In another embodiment, the pharmaceutically acceptable excipient consists essentially of Hydroxypropylcellulose, Edetate Disodium, Menthol, Butylated Hydroxytoluene, 2-(2-ethoxyethoxy)ethanol, Isopropyl Alcohol, Propylene Glycol, Glycerin, Lactic Acid, and Sodium Chloride.

In one embodiment, the combining comprises mixing. The mixing may be accomplished by various means, including flocculation, wetting, levigation, trituration, stirring, blending, homogenizing, sonication, injection, countercurrent exchange, impinging jet mixing, expansion of a supercritical fluid, and milling. As used herein, “levigation” is defined as the grinding to a powder of a moist or hard substance, or the mixing of a solid or particulate substance together with a solvating or wetting agent, thereby intimately mixing or coating the solid or particulate with the solvating or wetting agent.

In another embodiment, the invention provides methods for stabilizing an alkylating agent comprising: (a) providing a non-aqueous flowable ointment or cream, wherein the non-aqueous flowable ointment or cream does not include petrolatum, ethanol, or acetone; (b) combining an alkylating agent or pharmaceutically acceptable HX salt of an alkylating agent with a solvent that does not include petrolatum, ethanol, or acetone; and (c) combining the non-aqueous flowable ointment or cream of step (a) with the alkylating agent or pharmaceutically acceptable HX salt of the alkylating agent in the solvent of step (b).

In one embodiment, the method produces a composition of alkylating agent or pharmaceutically acceptable HX salt of the alkylating agent having a duration of activity from about 3 months to about 3 years. In another embodiment, the method produces a composition of alkylating agent or pharmaceutically acceptable HX salt of the alkylating agent having a duration of activity from about 3 months to about 5 years.

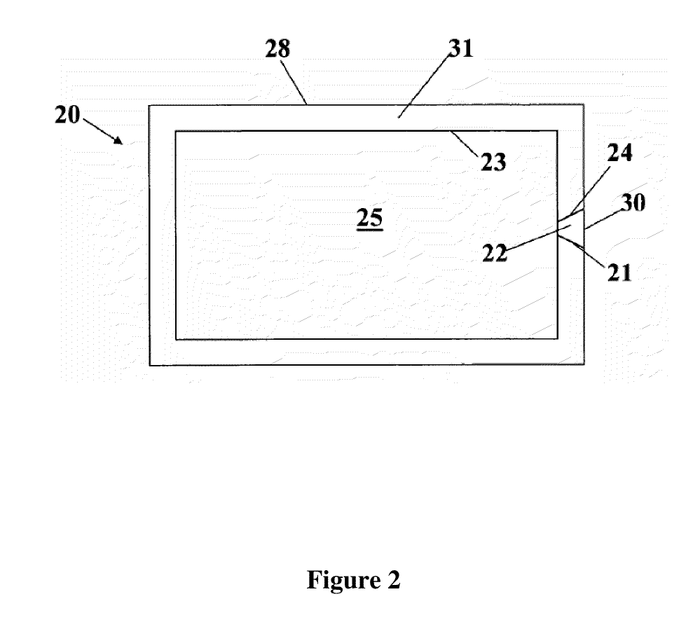

The type of container in which the compositions of the invention are stored may also affect the stability of the alkylating agents. The inventors disclose that lower volume containers having from about 0.01 to about 0.2 ml, from about 0.1 to about 0.5 ml, or from about 0.1 to about 1 ml may advantageously be used to provide from 1 to 10 applications of the pharmaceutically acceptable alkylating agent (such as Nitrogen Mustard.HCl) over a shorter period of use than the 50 ml flip top plastic cylinders, so that lower amounts of nucleophiles such as ambient water or other ambient nucleophiles such as methanol or ethanol may be introduced into the lower volume containers than when the flip top plastic cylinder is opened to the ambient environment over a period of 100 to 1000 applications. The inventors anticipate decreased decomposition of the free base form of nitrogen mustard or its HX salt, when the nitrogen mustard is contained in lower volume containers intended for from about 1 to 10 applications. In theory, the chlorides of the free base form of the nitrogen mustard or its HX salt may be displaced by nucleophilic attack, such as by water or ethanol, resulting in substitution of the Cl by an OH. Said decomposition of the free form of the nitrogen mustard or its HX salt may be avoided by isolating the nitrogen mustard from traces of water, ethanol or other nucleophiles in the environment. An apparatus 20, as depicted in FIG. 2, infra, depicts this smaller volume container.

FIG. 2 depicts a front cross-sectional view of the apparatus 20 for containing the compositions of the invention, comprising: a compartment 25 enclosed by a wall 31. The wall 31 comprises an outer surface 28 and an inner surface 23, ends 24 and 21, and opening 30. The first compartment 25 may be charged with the essentially completely uniform mixture of the composition through the opening 30. The opening 30 may be closed with plug 22. The plug 22 may be made of the same material as the wall 31, or a lower melting plastic or wax material.

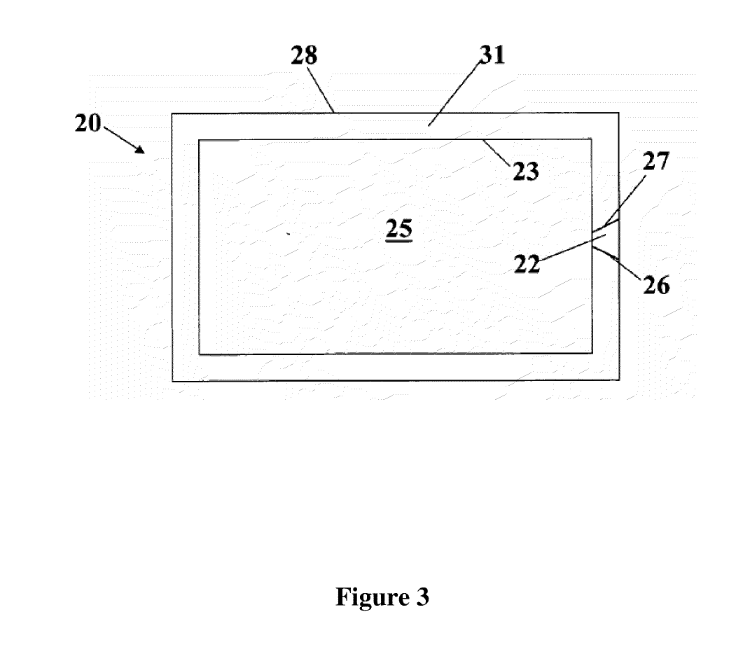

FIG. 3 depicts the apparatus 20, after forming heat seals 26 and 27 by heating the plug 22 and the ends 21 and 24 to their melting points, wherein heating physically and mechanically couples ends 21 and 24 to form mechanically strong heat seals 26 and 27.

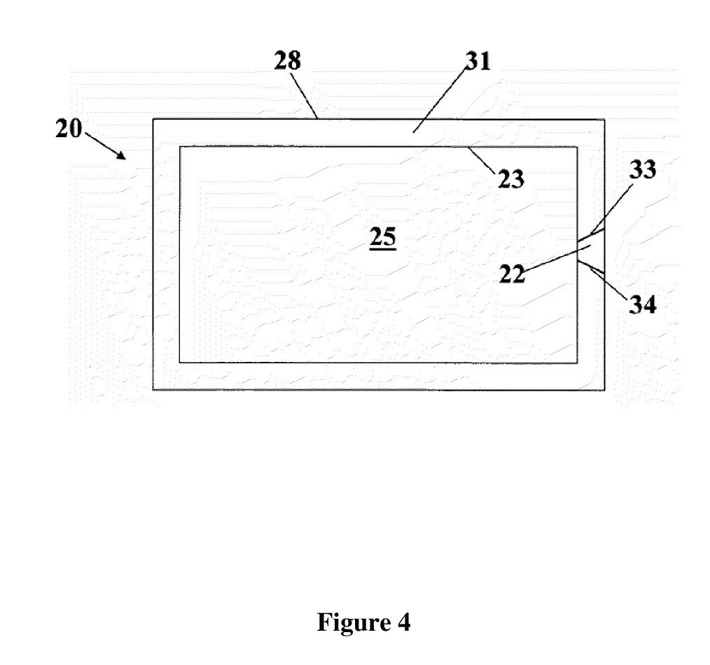

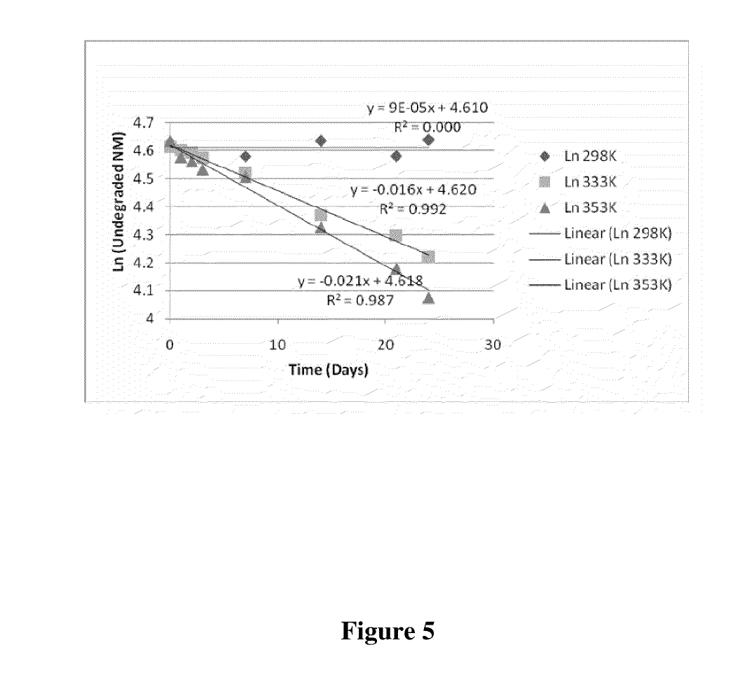

FIG. 4 depicts the apparatus 20, after forming serrated perforations 33 and 34 in the plug 22 using a crimping tool or other appropriate device for forming serrated perforations 33 and 34. The serrated perforations 33 and 34 weaken the heat seals 21 and 24 so that they become mechanically less strong, resulting in a tear line for removal of the plug 22 by the patient seeking to apply the composition to a diseased area or area for treatment. In one embodiment, a person wishing to apply the composition to the diseased area or area for treatment may remove plug 22 from the wall 31 of the apparatus 20, restoring the opening 30 in the wall 31 so squeezing or applying pressure to the wall 31 reduces the volume of the apparatus 20, resulting in the composition flowing outward through opening 30 to be applied to the diseased area or area for treatment.

In one embodiment, the nitrogen mustard alkylating agent is present in the composition in the compartment 25 in an amount of about 1 mg to about 2000 mg per 100 mL of the composition; about 10 mg to about 40 mg per 100 mL of the composition; or about 15 mg to about 30 mg per 100 mL of the composition. An orifice or opening 30 may be made by forming an opening in the outer wall 31, through which opening 30 the composition is then provided by prescription of a physician for treatment of the patient.

The outer wall 31 of apparatus 20 is impermeable to the mixture of step g) or step g), supra, and/or the reconstituted nitrogen mustard solutions. The wall 31 may be made from elastomeric materials including ethylene/propylene copolymers, ethylene/ethylacrylate copolymers, ethylene/vinyl acetate copolymers, silicone elastomers, medical-grade polydimethylsiloxanes, neoprene rubber, polyisobutylene, chlorinated polyethylene, polyvinyl chloride, vinylchloride-vinyl acetate copolymer, polymethacrylate polymer (hydrogel), polyvinylidene chloride, poly(ethylene terephthalate), butyl rubber, epichlorohydrin rubbers, ethylene-vinyl alcohol copolymer, ethylenevinyloxyethanol copolymer; silicone copolymers, polysiloxane-polycarbonate copolymers, polysiloxane-polyethyleneoxide copolymers, polysiloxane-polymethacrylate copolymers, polysiloxane-polymethacrylate copolymers, polysiloxane-alkylene copolymers polysiloxane-ethylene copolymers, polysiloxane-alkylenesilane copolymers, polysiloxaneethylenesilane copolymers, cellulose polymers, methyl cellulose, ethyl cellulose, hydroxypropyl methyl cellulose, cellulose esters, polycarbonates, polyesters, polytetrafluoroethylene, starches, gelatins, natural gums, synthetic gums, and combinations thereof.

In one particular embodiment, the invention provides methods for stabilizing nitrogen mustard alkylating agents by combining them with the pharmaceutically acceptable excipient 2-(2-ethoxyethoxy)ethanol.

Nitrogen mustard alkylating agents, such as MCHCl, are recognized by those of ordinary skill in the art to rapidly degrade to inactive products in the presence of base, water and many pharmaceutical excipients, including primary alcohols such as ethanol. As such, most MCHCl topical formulations currently employed for the treatment of dermatological conditions are composed of MCHCl dispersed in hydrophobic excipients, such as petrolatum, mineral oil and other lipophilic substances. These products tend to be stiff, have a high skin drag, and leave an adhesive, greasy layer on the skin that may also stain clothing or rub off on others, characteristics not generally acceptable to patients.

Formulation of these products is generally accomplished by mixing MCHCl powder directly into these viscous substances. Thus, the homogeneous incorporation and distribution of the dry powder into the oleaginous vehicle is complicated by clumping, sticking and caking of the dry powder in the vehicle, thereby requiring extensive mixing and homogenizing, as well as levigation and wetting agents not necessarily desirable in the final product. In addition, current formulation methods require repeated handling of the highly poisonous MCHCl powder, which is easily swept up and dispersed in the air, thereby posing a serious contamination risk for both personnel and the manufacturing facility.

Alternative topical formulations employing less lipophilic and amphipathic excipients have been explored. These excipients include 2-(2-ethoxyethoxy)ethanol, marketed under various trade names, including 2-(2-ethoxyethoxy)ethanol and diethylene glycol monoethyl ether. Although 2-(2-ethoxyethoxy)ethanol has served as an effective vehicle for many drugs, it is a primary alcohol, and many commercial forms of this excipient contain significant amounts of water and other potentially nucleophilic and solvolytic impurities, including the primary alcohols 2-methoxyethanol and 2-ethoxyethanol.

The inventors report, however, that MCHCl may be dispersed in 2-(2-ethoxyethoxy)ethanol across a wide range of concentrations while remaining stable for extended periods of time across a wide temperature range, even though 2-(2-ethoxyethoxy) ethanol is a primary alcohol. MCHCl remains stable when dispersed in commercial 2-(2-ethoxyethoxy)ethanol containing 0.1% w/w or more of water, which generally promotes solvolysis of this nitrogen mustard. The inclusion of stabilizing agents, such lactic acid or sodium chloride, has no significant effect on nitrogen mustard stability in the dispersion, and is not necessary for preparing a stable dispersion. Notably, MCHCl remains completely stable when dispersed in 2-(2-ethoxyethoxy) ethanol for over three months at room temperature. Significant degradation of MCHCl in the dispersion is only noted at elevated temperatures. Indeed, less than ten percent of the MCHCl in such a dispersion degraded after storage at temperature as high as 80° C. for an entire week. (See Example 7 below).

That MCHCl remains stable in the presence of the primary alcohol 2-(2-ethoxyethoxy)ethanol and water, and without stabilizers, preservatives or cooling agents, is particularly surprising, as it is known that MCHCl readily undergoes rapid solvolysis and nucleophilic substitution when in contact with many primary alcohols, water and mixtures thereof. This unexpected result, wherein MCHCl, known to be readily degraded in the presence of water and primary alcohols, and at elevated temperatures, remained stable in a 2-(2-ethoxyethoxy)ethanol dispersion for long periods of time at practical working temperatures, is neither obvious nor predictable, and underscores the novelty and utility of such a dispersion and its applications and uses. Indeed, since the pKa of 2-(2-ethoxyethoxy)ethanol and the alkoxy alcohol impurities in 2-(2-ethoxyethoxy)ethanol is lower than that of ethanol, they are theoretically even more nucleophilic than ethanol. Nevertheless, MCHCl, normally labile when exposed to such compounds, is quite stable in this dispersion.

### IV. METHODS FOR TREATING SKIN DISORDERS

In one embodiment, the invention encompasses methods for treating a skin disorder comprising topically administering to a subject in need thereof a stable composition comprising an effective amount of an alkylating agent or a pharmaceutically acceptable salt, solvate, or prodrug thereof, and at least one pharmaceutically acceptable excipient.

In one embodiment, the alkylating agent is a nitrogen mustard. In another embodiment, the alkylating agent is a nitrogen mustard of Structure (VII), (VIII), (IX), (X), (XII), (XIII), (XIV), (XV), (XVI), (XVII), (XVIII) or (XIX). In another embodiment, the nitrogen mustard is bis-(2-chloroethyl)ethylamine, bis-(2-chloroethyl)methylamine, or tris-(2-chloroethyl)amine. In another embodiment, the nitrogen mustard is bis-(2-chloroethyl)methylamine.

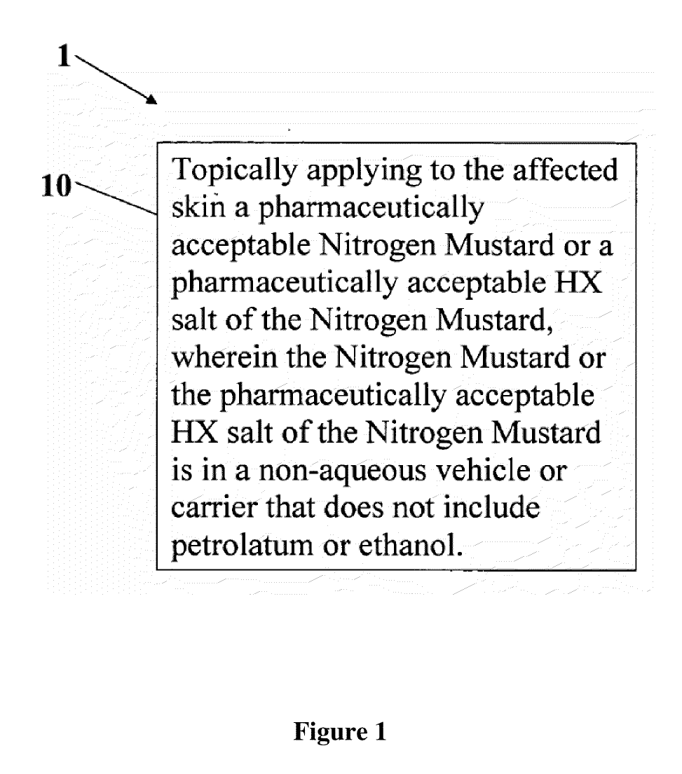

An illustrative embodiment of the method is depicted in FIG. 1. FIG. 1 depicts a method 1, for treating a person with a skin disorder, comprising: a step 10, topically applying to the affected skin a nitrogen mustard or a pharmaceutically acceptable FIX salt of the Nitrogen Mustard, wherein the nitrogen mustard or the pharmaceutically acceptable 1-DC salt of the Nitrogen Mustard is in a non-aqueous vehicle or carrier that does not include petrolatum, ethanol, or acetone.

In one embodiment, the skin disorder is a T-cell mediated skin disorder. In one embodiment, the T-cell mediated skin disorder is psoriasis, actinic keratosis, cutaneous T-cell lymphoma, cutaneous B-cell lymphoma, mycosis fungoides, alopecia, alopecia greata, or vitiligo.

In one embodiment, the skin disorder is psoriasis, eczema, actinic keratosis, lupus, sarcoidosis, alopecia, alopecia greata, cutaneous T-Cell lymphoma, i.e., mycosis fungoides, lymphoreticular neoplasia, pleural or peritoneal effusions, cutaneous B-cell lymphoma, pseudolymphoma of the skin, squamous cell carcinoma, basal cell carcinoma, bronchogenic carcinoma, malignant melanoma, lymphosarcoma, chronic lymphocytic leukemia, polycythemia vera, lymphomatoid papulosis, Mucha-Habberman's disease (PLEVA), or vitiligo.

In one embodiment, patients having mycosis fungoides (MF) topically treated with nitrogen mustard compounded into a polypropylene glycol (PPG, molecular weight from about 300 to about 2500), propylene glycol (PG), polyethyleneglycol (PEG, molecular weight from about 100 to about 1000) or ethylene glycol ointment or cream showed no evidence of any systemic toxicities.

Table 1 below provides a summary of topical treatment of patients having MF with a nitrogen mustard composition A, as described in Table 2 below (containing propylene glycol (PG)), including response rates and toxicities.

TABLE 1
Composition A in Mycosis Fungoides (MF): Summary of Clinical Outcomes and
Toxicities

| | | | | | | % Hyper- | |
|---|---|---|---|---|---|---|---|
| No. of | Follow- | | | % Complete | % Partial | sensitivity | % Systemic |
| patients | up (yrs) | Vehicle | Dose | response | Response | reactions | Toxicities* |
| 14 | <1 | Propylene | 10 mg % | 36% | 42% | 7% | 0% |
| | | glycol | topically | | | | |
| | | | applied once | | | | |
| | | | daily | | | | |

*Systemic toxicities monitored by serial History & Physicals laboratory studies

### Systemic Absorption

There is no evidence of any clinically significant systemic absorption of topically applied nitrogen mustard. No systemic toxicities from percutaneous absorption have been observed in long-term topical nitrogen mustard use in MF.

Genetic toxicity—No genetic toxicity has been observed with the use of topical nitrogen mustard application. This is best documented in a study that demonstrated no effect on sister-chromatid exchanges in the peripheral blood lymphocytes of CTCL-MF patients assayed before and after topical nitrogen mustard treatment.

Bone Marrow Suppression—No evidence of bone marrow suppression (anemia, leukoopenia or thrombocytopenia) has reported with long term use of topical nitrogen mustard, based on serial monitoring of the complete blood count.

Heptatotoxicity—No evidence of hepatotoxicty has reported with long term use of topical nitrogen mustard, based on serial monitoring of peripheral blood liver function tests.

Nephrotoxicity—No evidence of nephrotoxicity has been reported with long term use of topical nitrogen mustard, based on serial monitoring of peripheral blood renal function tests.

### Environmental Contamination

Minimal evidence of environmental contamination has been demonstrated with topical nitrogen mustard use.

### Cutaneous Side Effects

Hyperpigmentation—resulting from the direct melanogenic effects of nitrogen mustard, has been reported in a large percentage of treated patients. The hyperpigmentation is reversible and decreases gradually in most patients, even if topical therapy is continued.

Contact dermatitis—is a common complication of topical nitrogen mustard application. An irritant contact dermatitis is most common and can be seen in up to 25% of individuals using topical nitrogen mustard ointment, particularly if used in sensitive areas such as the face or skin folds. Allergic contact dermatitis is also observed with topical nitrogen mustard use.

Immediate-type (urticarial) reactions—are rare.

Allergic contact dermatitis—from delayed-type hypersensitivity (DTH) reactions is more common and appears to be dose-dependent. Higher concentrations of aqueous preparations are associated with a DTH frequency of 10-67%. Desensitization with lower concentrations of nitrogen mustard has been successfully employed in patients with DTH reactions to nitrogen mustard. The use of a lower concentration ointment preparation dramatically reduces the incidence of DTH reactions. Stanford University reported 0% DTH reactions in patients using nitrogen mustard ointment for the first time and an 8% frequency of DTH in patients with a previous history of HN hypersensitivity, in their series utilizing a nitrogen mustard ointment preparation.

### Pediatric Use

Topical nitrogen mustard has been reported to be used in children and adolescents (<18 years) without any significant differences in toxicities than in adults.

### Use in Pregnancy

Despite the lack of evidence of percutaneous absorption of topical nitrogen mustard, the use of topical nitrogen mustard has historically been avoided in pregnant and nursing women.

### Cutaneous Carcinogenesis

There are no reports of a significantly increased incidence of squamous cell carcinoma (SCC) of the skin with prolonged use of topical nitrogen mustard. Several groups have reported an approximately 10% (4%-14%) frequency of SCC in CTCL-MF patients using topical nitrogen mustard and suggest a potential risk of epidermal carcinogenesis. These retrospective studies, however, do not account for confounding variables, such as CTCL-MF associated risk for second malignancies, prior therapies (e.g. radiation therapy to the skin), and do not have adequate control groups.

The rationale for the use of topical nitrogen mustard for the treatment of vitiligo lies in the clinical and experimental observations that nitrogen mustard produces cutaneous hyperpigmentation not associated with an inflammatory response. It has long been observed that the treatment of MF with topical nitrogen mustard produces hyperpigmantation. The same phenomenon has been reported in nitrogen mustard treatment of psoriasis. Indeed, the inventors disclose successful regimentation of vitiligo treated with topical nitrogen mustard has been demonstrated. Supporting the direct effect of nitrogen mustard on melanogenesis, the pigmentation of hairless mice in response to topical nitrogen mustard. In humans, ultra structure studies demonstrate topical NM increases melanosome numbers and distribution without toxic effects to epidermal microenvironment.

In one embodiment, the composition of alkylating agent is topically administered to humans or animals in the form of a sterile solution or suspension that contains a suitable quantity of alkylating agent. In one embodiment, the composition comprises an effective amount of alkylating agent. In some embodiments, the topical solution or suspension is incorporated in a slow release non-aqueous matrix for administering transdermally.

In one embodiment, the composition is topically administered to the subject once daily. In another embodiment, the composition is topically administered to the subject twice daily. In another embodiment, the composition is topically administered to the subject every other day, every third day, every fourth day, every fifth day, every sixth day, or once weekly.

In some embodiments, the effective amount of alkylating agent is about 1 ng to about 40 mg per 1.9 m² per day, about 10 ng to about 10 mg per 1.9 m² per day, or about 100 ng to about 4 mg per 1.9 m² per day. In other embodiments, the effective amount of alkylating agent is about 0.5 ng to about 20 mg per m² per day, about 5 ng to about 5 mg per m² per day, or about 50 ng to about 2 mg per m² per day.

In other embodiments, the effective amount of alkylating agent is about 1 ng to about 40 mg per 60 kg per day, about 10 ng to about 10 mg per 60 kg per day, or about 100 ng to about 4 mg per 60 kg per day. In other embodiments, the effective amount of alkylating agent is about 0.02 ng to about 0.7 mg per kg per day, about 0.2 ng to about 0.2 mg per kg per day, or about 1.7 ng to about 0.07 mg per kg per day.

In some embodiments of the methods, the composition contains a vehicle or carrier that ameliorates skin irritation that can result from administration of the nitrogen mustard, pharmaceutically acceptable salt of the nitrogen mustard, or nitrogen mustard prodrug. In some embodiments, the composition is effective to treat the skin disorder, but does not cause hypersensitivity reactions.

In another embodiment, the compositions of the invention can be used as adjunct therapy in combination with existing therapies, such as for hyperthermia or in the management of cancer treatment in patients having cancer. In one embodiment, the invention encompasses a method for treating a T-cell mediated skin disorder comprising administering a nitrogen mustard and another therapeutic agent.

In some embodiments, topical administration of a composition of the invention is effective to produce a response to treatment. In one embodiment, response to treatment is determined based on a controlled trial. Typically, the controlled trial is a double-blinded trial. The double-blinded trial may be performed with or without biases.

In some embodiments, the response rate in a group of human patients is greater than about 60% after at least six months of treatment with a composition of the invention. In other embodiments, the response rate in a group of human patients is greater than about 65%, greater than about 70%, greater than about 75%, greater than about 80%, greater than about 85%, greater than about 90%, or greater than about 95% after at least six months of treatment with a composition of the invention.

In other embodiments, the response rate in an intent-to-treat group of human patients is greater than about 50% after treatment with a composition of the invention. In other embodiments, the response rate in an intent-to-treat group of human patients is greater than about 55%, greater than about 60%, greater than about 65%, greater than about 70%, greater than about 75%, greater than about 80%, greater than about 85%, greater than about 90%, or greater than about 95% after treatment with a composition of the invention.

In other embodiments, the response rate in a group of human patients is greater than about 55% upon two months of treatment. In other embodiments, the response rate in a group of human patients is greater than about 60%, greater than about 65%, greater than about 70%, greater than about 75%, greater than about 80%, greater than about 85%, greater than about 90%, or greater than about 95% upon two months of treatment with a composition of the invention.

In some embodiments, the time to achieve a response rate of 50% in a group of human patients is about 40 weeks or less. In other embodiments, the time to achieve a response rate of 50% in a group of human patients is about 35, about 30, about 25, about 20, about 15, about 10, or about 5 weeks or less.

In some embodiments, topical administration of a composition of the invention is more effective in producing a response to treatment than a reference composition consisting of an equivalent amount of the nitrogen mustard, a hydrophobic carrier, and an organic solvent. Typically, the reference composition consists of a nitrogen mustard in aquaphor (i.e., petrolatum, mineral oil, ceresin, lanolin, panthenol, glycerin, bisabolol, and ethanol).

In one embodiment, the response rate achieved in a group of human patients upon application of a composition of the invention is greater than the response rate achieved upon application of a reference composition. In another embodiment, the response rate upon application of a composition of the invention is at least about 5%, at least about 10%, at least about 15%, at least about 20%, at least about 25%, at least about 30%, at least about 35%, or at least about 40% greater than the response rate achieved upon application of the reference composition.

In another embodiment, the time to achieve a response rate of 50% in a group of human patients upon application of a composition of the invention is less than the time to achieve a response rate of 50% upon application of a reference composition. In another embodiment, the time to achieve a response rate of 50% in a group of human patients is at least about 10%, at least about 15%, at least about 20%, at least about 25%, at least about 30%, at least about 35%, or at least about 40% less than the time to achieve a response rate of 50% achieved upon application of the reference composition. In some embodiments, the time to achieve a response rate of 50% in a group of human patients is at least about 5, at least about 10, at least about 15, or at least about 20 weeks less than the time to achieve a response rate of 50% achieved upon application of the reference composition.

Typically, the safety of the compositions of the invention upon application is comparable to the safety observed upon application of the reference composition. In one embodiment, the percent occurrence of an adverse event in a group of human patients upon application of the composition is substantially equal to the percent occurrence of the adverse event upon application of the reference composition. Adverse events include, but are not limited to, skin toxicity, such as allergic contact dermatitis, irritant contact dermatitis, or hypersensitivity, or skin cancer.

### V. OTHER USES

The compositions of the invention have a number of additional uses and applications, such as formulation aids and as concentrated sources of alkylating agents for dilution and incorporation into a variety of dispersed systems and pharmaceutical products.

In one embodiment, the invention encompasses methods of using the above-described compositions as a formulation aid, as and as a means of storing, transporting, and dispensing discrete quantities of an alkylating agent for use in pharmaceutical formulations and other preparations. In one embodiment, the composition comprises an alkylating agent dispersed in 2-(2-ethoxyethoxy)ethanol.

In another embodiment, the invention encompasses an alkylating agent or agents dispersed in 2-(2-ethoxyethoxy)ethanol for use as a formulation aid, where said formulation aid is employed as a dispersion of a pharmaceutically acceptable alkylating agent or mixture of alkylating agents for subsequent dispersion and dilution into a bulk pharmaceutical product during the formulation and manufacture of said product.

As a formulation aid, the composition can serve as a pre-solvated, pre-dispersed form of an alkylating agent for ready dispersion and homogeneous mixing into a pharmaceutical formulation or other preparation, such as a solution, a suspension, an ointment, a cream, a lotion, a plaster, a spray, a colloid and a paste. Such a pre-dispersed form of an alkylating agent, already de-gassed and solvated, facilitates homogeneous mixing into such dosage forms while minimizing or eliminating clumping, flocculation, agglomeration, sticking and caking of alkylating agents.

The composition can be stored in any suitable container, such as a jar, a bottle, a flask, a bag, a collapsible bag, a bladder, a syringe, a collapsible tube or a drum. Said container might also have an appropriate dispensing port, such as a mouth, a spigot, a valve, a syringe port, and a pump. Said container might also be pressurized, or be charged by or attached to an inert gas source, such as dry nitrogen or helium, in order to further maintain stability of the dispersion and replace the dispensed volume of the dispersion with inert gas.

In another embodiment, the invention encompasses a method of formulating a pharmaceutical product, a component of which is at least one hydrolytically unstable alkylating agent(s), comprising: providing a formulation aid, wherein said formulation aid is a pre-solvated or pre-dispersed form of the alkylating agent; and dispersing the formulation aid into a pharmaceutical formulation or other preparation, wherein the formulation aid and the pharmaceutical formulation are substantially homogeneous. In one embodiment, the alkylating agent is a nitrogen mustard. In one embodiment, the formulation aid is 2-(2-ethoxyethoxy)ethanol.

In another embodiment, the invention encompasses a method for preparing a composition comprising an alkylating agent or a pharmaceutically acceptable salt, solvate, or prodrug thereof, and a pharmaceutically acceptable excipient, comprising: combining the alkylating agent or pharmaceutically acceptable salt, solvate, or prodrug thereof and the pharmaceutically acceptable excipient.

Having described the invention with reference to certain embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Preparation of a Topical Ointment Comprising Bis-(2-chloroethyl)methylamine Hydrochloride in a Pharmaceutically Acceptable Vehicle or Carrier

(a) Composition of the Topical Ointment

The components/composition of the topical ointment is provided in the following Table 2.

TABLE 2
Composition A

| | Amount per | | |
|---|---|---|---|
| Ingredient | 100 mL | | Percent |
| PPG, PG, PEG, or EG, USP | 15-60 | mL | 15-60% |
| 2-(2-ethoxyethoxy)ethanol | 15-60 | mL | 15-60% |
| Hydroxypropylcellulose, NF (1500 cps) | 0.75 | g | 0.75% |
| Menthol crystals, USP | 0.08 | g | 0.08% |
| Butylated hydroxytoluene, NF | 0.05 | g | 0.05% |
| (BHT) | | | |
| Glycerin, USP | 12.75 | mL | 12.75% |
| Edetate disodium, USP | 0.05 | g | 0.05% |
| Decyl methyl sulfoxide | 0.125 | g | 0.13% |
| Kris-Ester 236 liquid | 5 | g | 5.00% |
| Alcohol anhydrous 100% SDA 3A | 2.175 | mL | 2.18% |
| Bis-(2-chloroethyl)methylamine | 0.001-2 | g | 0.001-2.0% |
| HCl^{b} | | | |

^{a}Available from Merck & Co., West Point, PA 19486

(b) Manufacturing

The topical ointment may be manufactured according to the following general procedure:

### Preparation of the Ointment:

1. All dry excipient ingredients are assembled and weighed out according to the formula in Table 2 and placed in an appropriate vessel. Hereinafter, an excipient is an inert substance which is added to the free base form of the nitrogen mustard or its pharmaceutically acceptable HX salt to provide bulk. Hereinafter, the dry excipient ingredients are indicated as being added as solid weight, such as gram, i.e. gm.
2. Particle sizes of the dry material are reduced to a uniform size through titration.
3. Polypropylene glycol (PPG), propylene glycol (PG), polyethylene glycol (PEG) or ethylene glycol (EG) from about 15 to about 60 percent by weight is then added via the principle of geometric dilution to form a smooth paste. Once a smooth paste is achieved, the propylene or ethylene glycol continues to be added until a volume that retains a flow like quality is obtained.
4. The entire contents are then transferred to a large beaker. A spin bar is added and the beaker is placed on a magnetic stirring plate and mixing is begun.
5. As the mixture continues to spin, glycerin is added. While the mixture spins, the original vessel is washed with from about 15 to about 60 percent by weight ethoxy digycol and the contents of the vessel are added to the spinning mixture in the beaker.
6. After the 2-(2-ethoxyethoxy)ethanol is added, kris-ester is added to the spinning mixture. This mixture then is spun for approximately one to two hours. After the spinning is finished the mixture is covered and left to sit over-night.
7. The next day the mixture is mixed with a high shear mixer to a uniform consistency with minimal to no air. Air and moisture may be removed during mixing by applying a vacuum from about 0.01 to about 0.1 torr. The mixture is then brought to ambient pressure by adding dry nitrogen.

### Adding the Nitrogen Mustard:

8. The appropriate concentration and amount of Nitrogen mustard is reconstituted with absolute alcohol (200 proof) then added to the appropriate amount of non-aqueous vehicle or carrier, wherein the non-aqueous vehicle or carrier does not include petrolatum or ethanol and mixed to a uniform consistency via agitation for 60-90 seconds For example, in an embodiment, a concentration in mg/ml of the pharmaceutically acceptable Nitrogen Mustard.HCl in the non-aqueous vehicle or carrier that does not include petrolatum or ethanol is advantageously from about 1 mg of Nitrogen Mustard.HCl per 100 ml of non-aqueous vehicle to about 2000 mg of Nitrogen Mustard.HCl per 100 ml of non-aqueous vehicle. In an embodiment, a concentration in mg/ml of Nitrogen Mustard.HCl in a non-aqueous vehicle or carrier that does not include petrolatum or ethanol is advantageously from about 10 mg of Nitrogen Mustard.HCl per 100 ml of non-aqueous vehicle or carrier that does not include petrolatum or ethanol to about 40 mg of Nitrogen Mustard.HCl per 100 ml of non-aqueous vehicle or carrier that does not include petrolatum or ethanol. In an embodiment, a concentration in mg/ml of Nitrogen Mustard.HCl used in a non-aqueous vehicle or carrier that does not include petrolatum or ethanol is advantageously from about 15 mg of Nitrogen Mustard.HCl per 100 ml of non-aqueous vehicle or carrier that does not include petrolatum or ethanol to about 30 mg of Nitrogen Mustard.HCl per 100 ml of non-aqueous vehicle or carrier that does not include petrolatum or ethanol.
9. This mixture is then poured into a 50 ml flip top plastic cylinder and shipped to the appropriate patient.

### Clean-Up:

10. All vessels used in the process are placed in a Sodium Thiosulfate-Sodium Bicarbonate aqueous bath. Contents are left in the bath for 2 hours and then the washed. The bath is then discarded by normal means. Note: Sodium Thiosulfate reacts with nitrogen mustard to create an innocuous, safe mixture that can be discarded by normal means.

### Example 2

### Preparation of a Topical Ointment Comprising Bis-(2-chloroethyl)methylamine Hydrochloride in a Pharmaceutically Acceptable Vehicle or Carrier

(a) Composition of the Topical Ointment

The components/composition of the topical ointment is provided in the following Table 3.

TABLE 3
Composition B

| | Parts per 100 Parts |
|---|---|
| | by Weight of a |
| Ingredient^{a} | Topical Ointment |
| Dimethicone or Cyclomethicone | 10-60 |
| 2-(2-ethoxyethoxy)ethanol | 10-16 |
| Hydroxypropylcellulose, NF (1500 cps) | 0-15 |
| Menthol crystals, USP | 0-1 |
| Butylated hydroxytoluene, NF (BHT) | 0-1 |
| Glycerin, USP | 1-2 |
| Edetate disodium, USP | 0-0.05 |
| Decyl methyl sulfoxide | 0-0.125 |
| Kris-Ester 236 liquid | 0-5 |
| Anhydrous secondary or tertiary | 1-20 |
| alcohol | |
| Bis-(2-chloroethyl)methylamine HCl^{b} | 0.001-2.0 |
| CₙH₍₂ₙ₊₂₎COOH, (n = 1-6) | 0.01-15 |

^{a}Slight overages of the ingredients may be used as required to offset losses during manufacture.
^{b}Available from Merck & Co., West Point, PA 19486

(b) Manufacturing

The topical ointment may be manufactured according to the following general procedure:

### Preparation of the Dimethicone or Cyclodimethicone Ointment:

1. All dry excipient ingredients are assembled and weighed out according to the formula in Table 3 and placed in an appropriate vessel.
2. Particle sizes of the dry material are reduced to a uniform size through titration.
3. Dimethicone or cyclomethicone from about 10 to about 60 percent by weight is then added via the principle of geometric dilution to form a smooth paste. Once a smooth paste is achieved, the Dimethicone or cyclomethicone continues to be added until a volume that retains a flow like quality is obtained.
4. The entire contents are then transferred to a large beaker. A spin bar is added and the beaker is placed on a magnetic stirring plate and mixing is begun.
5. As the mixture continues to spin, glycerin is added. While the mixture spins, the original vessel is washed with from about 10 to about 16 percent by weight 2-(2-ethoxyethoxy)ethanol and the contents of the vessel are added to the spinning mixture in the beaker.
6. After the 2-(2-ethoxyethoxy)ethanol is added, from about 0.01-15 percent by weight of a pH modifier such as citric acid, lactic acid or aliphatic acids having a formula CₙH₍₂ₙ₊₂₎COOH, (n=1-6) is added to the spinning mixture. This mixture then is spun for approximately one to two hours. After the spinning is finished the mixture is covered and left to sit over-night.
7. The next day the mixture is mixed with a high shear mixer to a uniform consistency with minimal to no air. Air and moisture may be removed during mixing by applying a vacuum from about 0.01 to about 0.1 torr. The mixture is then brought to ambient pressure by adding dry nitrogen.
   Combining the Nitrogen Mustard and the Dimethicone or Cyclodimethicone Non-Aqueous Vehicle or Carrier that does not Include Petrolatum or Ethanol of Step 7) Above:

The nitrogen mustard and the dimethicone or cyclodimethicone non-aqueous vehicle or carrier may be combined by agitating for 60-90 seconds using a high shear mixer to mix 1) an appropriate amount of nitrogen mustard having been be reconstituted with an secondary or tertiary alcohol such as isopropyl alcohol, wherein ethanol has been rigorously excluded from the secondary or tertiary alcohol such as isopropyl alcohol, and 2) the appropriate amount of non-aqueous vehicle or carrier from step 7), supra. For example, in an embodiment, a concentration in mg/ml of the pharmaceutically acceptable Nitrogen Mustard.HCl in the non-aqueous vehicle or carrier is advantageously from about 1 mg of Nitrogen Mustard.HCl per 100 ml of non-aqueous vehicle to about 2000 mg of Nitrogen Mustard.HCl per 100 ml of non-aqueous vehicle. In another embodiment, a concentration in mg/ml of the pharmaceutically acceptable Nitrogen Mustard.HCl used in the non-aqueous vehicle or carrier is advantageously from about 10 mg of Nitrogen Mustard.HCl per 100 ml of non-aqueous vehicle or carrier to about 40 mg of Nitrogen Mustard.HCl per 100 ml of non-aqueous vehicle or carrier. In another embodiment, a concentration in mg/ml of the pharmaceutically acceptable Nitrogen Mustard.HCl used in the non-aqueous vehicle or carrier is advantageously from about 15 mg of Nitrogen Mustard.HCl per 100 ml of non-aqueous vehicle or carrier to about 30 mg of Nitrogen Mustard.HCl per 100 ml of non-aqueous vehicle or carrier.

This mixture is then poured into a 50 ml flip top plastic cylinder and shipped to the appropriate patient. The mixture advantageously has an essentially completely uniform consistency.

### Clean-Up:

All vessels used in the process are placed in a 5% w/v sodium bicarbonate-sodium thiosulfate aqueous bath. Contents are left in the bath for 2 hours and then the washed. The bath is then discarded by normal means. Note: Sodium Thiosulfate reacts with nitrogen mustard to create an innocuous, safe mixture that can be discarded by normal means.

### Example 3

### Preparation of a Topical Ointment Comprising Bis-(2-chloroethyl)methylamine Hydrochloride in a Pharmaceutically Acceptable Vehicle or Carrier

The topical ointment described in Example 1 or 2 may alternatively be manufactured according to the following general procedure:

### Preparation of the Ointment:

1. All dry (i.e., solid) excipient ingredients, except for hydroxypropylcellulose, are assembled and weighed out according to the formula in Table 2 or 3 and placed in appropriate containers.
2. All wet (i.e., liquid) excipient ingredients, except for glycerin and 2-(2-ethoxyethoxy)ethanol, are weighed out according to the formula in Table 2 or 3 and combined in a large vessel with all dry excipient ingredients. These excipient ingredients are mixed at low speed to form a uniform mixture (“Excipient Mixture”).
3. In a separate vessel, Glycerin and Hydroxypropyl cellulose are weighed out according to the formula in Table 2 or 3, combined in the vessel and mixed at low speed to form a uniform suspension (“Glycerin/HPC suspension”).
4. While maintaining constant low speed mixing, the Glycerin/HPC suspension is added directly to the Excipient Mixture in the large vessel to form a uniform ointment (or gel) base (“Ointment base”).

### Adding the Nitrogen Mustard:

5. The appropriate concentration and amount of Nitrogen Mustard is reconstituted with 2-(2-ethoxyethoxy)ethanol and added to the Ointment base. A final uniform Nitrogen Mustard product ointment is obtained by applying low speed constant mixing. Nitrogen Mustard product ointment is filled in appropriate size aluminum tube containers, crimp sealed to close and complete.

### Example 4

### Effect of Alcohol on Stability of Nitrogen Mustard Ointment Compositions

Two compositions of nitrogen mustard ointment (Compositions C and D) were produced to assess the differences in alcohols used for diluting the nitrogen mustard. Samples were tested at various temperature conditions (5, 25 and 40° C.) and assessed for stability with use of an HPLC assay. The formulations for Compositions C and D are encompassed by those described in Tables 4 and 5 below.

TABLE 4
Composition C

| | Percent by |
|---|---|
| | weight of the |
| Component | composition |
| MUSTARGEN ® (mechlorethamine hydrochloride and | 0.001-5% |
| sodium chloride) | |
| Hydroxypropyl cellulose, MXF | 0.01-5% |
| Edetate disodium dihydrate, USP | 0.01-1% |
| Menthol, USP | 0.01-1% |
| Butylated hydroxytoluene, USP | 0.01-10% |
| 2-(2-ethoxyethoxy) ethanol) (pharmaceutical grade) | 1-99% |
| Absolute alcohol (ethanol), USP | 1-50% |
| Propylene glycol, USP | 1-50% |
| Glycerin, USP | 1-50% |
| Citric acid, USP | 1-25% |
| Total | 100% |

TABLE 5
Composition D

| | | Percent by |
|---|---|---|
| | Quality | weight of the |
| Component | Standard | composition |
| Mechlorethamine hydrochloride | USP | 0.001-5% |
| Hydroxypropyl cellulose | NF | 0.01-5% |
| Edetate disodium (dihydrate) | USP | 0.01-1% |
| (DL) Menthol | USP | 0.01-1% |
| Butylated hydroxytoluene | NF | 0.01-10% |
| 2-(2-ethoxyethoxy)ethanol) | NF | 1-99% |
| Isopropyl alcohol | USP | 1-50% |
| Propylene glycol | USP | 1-50% |
| Glycerin | USP | 1-50% |
| Lactic acid (racemic) | USP | 1-25% |
| Sodium chloride | USP | 0.01-10% |
| Total | | 100% |

Composition C (˜15% absolute alcohol) yielded the following results when tested for stability at 1, 2 and 3 weeks post production (See Table 6).

TABLE 6
Label Strength of Composition C over time stored at various
temperatures in aluminum foil tubes.

| Day of | Storage | % MCHCl (or label |
|---|---|---|
| assessment | Temperature | strength) |
| 7 | 5° C. | 96.36 |
| | 25° C. | 81.37 |
| | 40° C. | 20.50 |
| 14 | 5° C. | 92.54 |
| | 25° C. | 68.44 |
| | 40° C. | 1.26 |
| 21 | 5° C. | 92.83 |
| | 25° C. | 58.85 |
| | 40° C. | −2.22 |

Composition D (˜15% isopropanol) yielded the following results when tested for stability at 10, 15, and 20 days post production (See Table 7).

TABLE 7
Label Strength of Composition D over time stored at various
temperatures in aluminum foil tubes.

| Day of | Storage | % MCHCl (or label |
|---|---|---|
| assessment | Temperature | strength) |
| 10 | 5° C. | 99.54 |
| | 25° C. | 98.42 |
| | 40° C. | 79.25 |
| 15 | 5° C. | 99.11 |
| | 25° C. | 95.75 |
| | 40° C. | 72.91 |
| 20 | 5° C. | 96.89 |
| | 25° C. | 93.07 |
| | 40° C. | 67.48 |

According to the data in Tables 6 and 7, absolute alcohol (ethanol) caused significant degradation of the mechlorethamine hydrochloride, while isopropyl alcohol did not.

After 18 months of storage at ambient temperature, i.e., between about 20° C. and 25° C., about 80% or more of the mechlorethamine hydrochloride remained in Composition D (in other words, there was about 20% or less loss of mechlorethamine hydrochloride due to degradation in Composition D).

### Example 5

### Effect of pH on Stability of Nitrogen Mustard Ointment Compositions

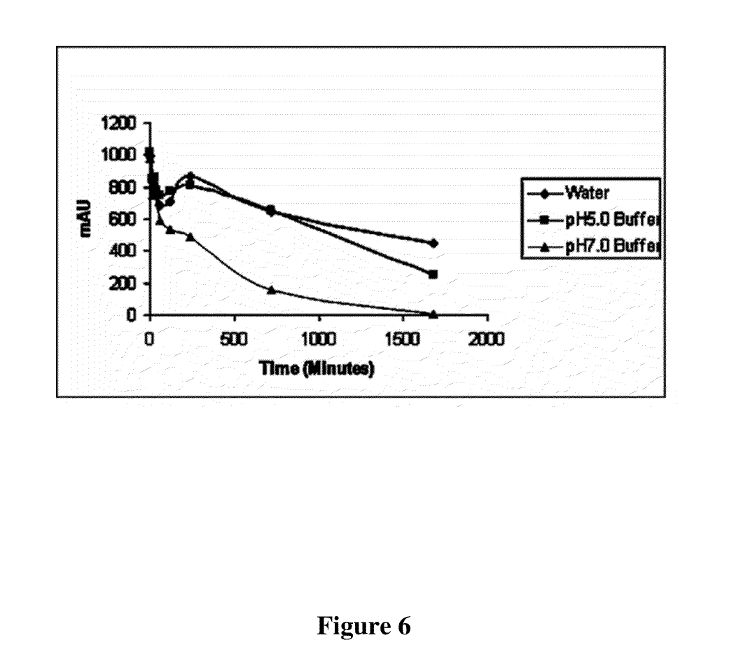

Mechlorethamine hydrochloride was dissolved in water, pH 5 buffer, and pH 7 buffer, and the degradation of the mechlorethamine hydrochloride was measured over a period of about 28 hours. The results are shown in FIG. 6.

As illustrated in FIG. 6, the mechlorethamine hydrochloride decomposes much faster in pH 7 buffer than in water (pH ˜5.2) or pH 5 buffer. The half life of the mechlorethamine hydrochloride in pH 7 buffer is about 2 hours, and the mechlorethamine hydrochloride decomposes totally after 28 hours. Thus, one can conclude that mechlorethamine hydrochloride decomposes faster in higher pH solutions, and that the presence of inorganic salts (such as in the pH 7 buffer) also can accelerate decomposition.

### Example 6

### Preparation of a Representative Dispersion

Mechlorethamine HCl (MCHCl) is readily dispersed in 2-(2-ethoxyethoxy) ethanol by depositing dry MCHCl powder into a suitable container, such as a flask, a vial or a bottle, adding 2-(2-ethoxyethoxy)ethanol, and dispersing the MCHCl by mixing, such as by stirring, sonicating or shaking.

Accordingly, a 0.5% w/w solution of MCHCl dissolved in 2-(2-ethoxyethoxy)ethanol is readily prepared by placing 25 mg of MCHCl powder in a 25 mL glass vial, adding 4.975 grams of 2-(2-ethoxyethoxy)ethanol, and stirring gently for 1 hour.

Equilibrium solubility experiments revealed that the solubility of MCHCl in 2-(2-ethoxyethoxy)ethanol is approximately 1.6% w/w.

### Example 7

### Stability of Mechlorethamine HCl Dispersed in 2-(2-ethoxyethoxy)ethanol at Various Temperatures

0.5% w/w solutions of mechlorethamine HCl (MCHCl) dissolved in 2-(2-ethoxyethoxy)ethanol (composition E); 2-(2-ethoxyethoxy)ethanol and NaCl (composition F); 2-(2-ethoxyethoxy)ethanol and 85% aqueous lactic acid (composition G); and 2-(2-ethoxyethoxy)ethanol, NaCl, and 85% aqueous lactic acid (composition H) were prepared, and aliquot parts were stored at various temperatures in glass vials sealed from the atmosphere. Percent MCHCl in each sample was measured by HPLC with a MS detector over time.

HPLC-MS was performed using the following parameters:

TABLE 8
High Performance Liquid Chromatography (HPLC) Parameters

| Device: | Waters ® Alliance 2695 Separation Module | | |
|---|---|---|---|
| Column: | Symmetry ® MS C18 column, 3.5 μ, 150 × 2.1 mm | | |
| Flow rate: | 0.2 mL/minute | | |
| Run time: | 60 minutes | | |
| Retention time: | ~17 minutes | | |
| Detector: | MS | | |
| Mobile phase: | A: 0.1% formic acid in water | | |
| | B: 0.1% formic acid in acetonitrile:water (95:5) | | |
| | Time | | |
| | (minutes) | % A | % B |
| Gradient: | 0 | 99% | 1% |
| | 3.10 | 1% | 99% |
| | 22 | 1% | 99% |
| | 41 | 99% | 1% |
| | 50 | 99% | 1% |
| | 60 | 99% | 1% |

TABLE 9
Mass Spectrometry (MS) Parameters

| | |
|---|---|
| Device: | Waters ® Micromass ESCi multimode ionization |
| Capillary: | 2.60 kV |
| Cone: | 21.0 V |
| Extractor: | 3 V |
| Rf Lens: | 0 V |
| Temperature: | Source temperature: 120° C. |
| | Desolvation temperature: 350° C. |
| Gas Flow: | Desolvation: 400 L/hour |
| | Cone: 50 L/hour |

Samples were prepared for HPLC analysis as follows: A nitrogen mustard sample (100 μL) was removed from the stored glass vials, combined with a 150 mM NaCl solution (100 μL), and mixed. The resulting solution was then combined with 750 mL of a solution of sodium diethyldithiocarbamate (DDTC) in sodium hydroxide (10 mg/mL of DDTC in 0.1M NaOH), mixed, and then incubated at 37° C. for 30 minutes. The resulting solution was then allowed to cool to room temperature and extracted three times with 1000 ethyl acetate. The combined organic extracts were evaporated to dryness under nitrogen at room temperature to form a residue. The residue was then reconstituted with 1000 μL of acetonitrile:water (20:80) with 0.1% v/v formic acid.

The results of the stability studies are presented in Tables 10 and 11 below.

TABLE 10
Stability of MCHCl compositions containing 2-(2-ethoxyethoxy) ethanol stored at
room temperature

| | Percent nitrogen mustard (MCHCl) remaining in samples | | | |
|---|---|---|---|---|
| Time | stored at ~25° C. (mean ± SD) | | | |
| (Weeks) | Composition E | Composition F | Composition G | Composition H |
| 0 | 102.31 ± 0.71 | 100.43 ± 0.29 | 98.40 ± 1.58 | 102.61 ± 0.53 |
| 1 | 97.31 ± 0.38 | 99.05 ± 0.24 | 103.68 ± 0.14 | 101.11 ± 0.15 |
| 2 | 102.94 ± 1.21 | 99.39 ± 0.52 | 103.57 ± 0.19 | 104.72 ± 0.07 |
| 3 | 97.43 ± 0.06 | 97.38 ± 0.04 | 95.25 ± 0.11 | 103.96 ± 0.41 |
| 4 | 103 ± 2.65 | 100.79 ± 0.37 | 100.55 ± 0.10 | 101.33 ± 0.10 |
| 5 | 99.59 ± 0.05 | 97.23 ± 0.31 | 99.98 ± 0.06 | 104.65 ± 0.27 |
| 6 | 100.03 ± 0.87 | 97.11 ± 2.56 | 97.08 ± 1.87 | 100.75 ± 5.38 |
| 7 | 100.15 ± 5.56 | 96.19 ± 2.75 | 96.74 ± 1.58 | 99.07 ± 3.98 |
| 8 | 98.68 ± 5.04 | 92.18 ± 1.45 | 95.49 ± 3.46 | 96.09 ± 3.65 |

Composition E: 0.5% MCHCl; 99.5% 2-(2-ethoxyethoxy) ethanol
Composition F: 0.5% MCHCl; 0.18% w/w NaCl; 99.32% 2-(2-ethoxyethoxy) ethanol
Composition G: 0.5% MCHCl; 3.65% w/w 85% aqueous lactic acid; 95.85% 2-(2-ethoxyethoxy) ethanol
Composition H: 0.5% MCHCl; 0.18% w/w NaCl; 3.65% w/w 85% aqueous lactic acid; 95.67% 2-(2-ethoxyethoxy) ethanol

Table 10 reveals that the inclusion of the stabilizing agents sodium chloride, lactic acid or both do not affect MCHCl stability in 2-(2-ethoxyethoxy)ethanol. Therefore, it can be concluded from Table 10 that MCHCl is stable in 2-(2-ethoxyethoxy)ethanol at room temperature for up to 8 weeks without the need for stabilizing agents, such as sodium chloride, lactic acid, or a combination thereof. Further, Table 10 illustrates that MCHCl is stable in 2-(2-ethoxyethoxy)ethanol at room temperature for up to 8 weeks without the addition of any antioxidant, such as BHT, EDTA, benzyl alcohol, or a paraben, as none of Compositions E to H contain an antioxidant. Further still, Table 10 illustrates that MCHCl is stable in 2-(2-ethoxyethoxy)ethanol at room temperature for up to 8 weeks, even in the presence of 0.5% by weight water, which is included in Compositions G and H in the form of aqueous lactic acid. Therefore, 2-(2-ethoxyethoxy)ethanol would serve as a useful formulation aid.

TABLE 11
Stability of MCHCl compositions containing 2-(2-ethoxyethoxy)
ethanol stored at elevated temperatures

Percent MCHCl remaining in
samples of Composition E
(mean ± SD)

| | Storage at 60° C. | Storage at 80° C. |
|---|---|---|
| Time | | |
| (Days) | | |
| 0 | 100.76 ± 0.91 | 103.13 ± 0.86 |
| 1 | 99.63 ± 3.66 | 97.20 ± 6.38 |
| 2 | 98.79 ± 2.03 | 95.85 ± 3.45 |
| 3 | 96.90 ± 4.9 | 92.98 ± 1.32 |
| Time | | |
| (Weeks) | | |
| 1 | 92.04 ± 4.06 | 90.66 ± 4.80 |
| 2 | 78.99 ± 0.99 | 75.73 ± 2.96 |
| 3 | 73.44 ± 4.91 | 65.36 ± 5.51 |
| 4 | 67.99 ± 3.96 | 58.94 ± 4.96 |

Table 11 reveals that, even at elevated temperatures, MCHCl remains stable in 2-(2-ethoxyethoxy)ethanol for significant periods of time, even at temperatures as high as 80 degrees Celsius for 1 week. This further underscores the utility and versatility of the unexpected observation that 2-(2-ethoxyethoxy)ethanol is a useful formulation aid for facilitating the storage, transport and dispensing of such alkylating agents in dispersion.

### Example 8

### Stability of Nitrogen Mustard Ointment Batches after Storage at Room Temperature

Three batches of ointment having 0.02% w/w mechlorethamine HCl according to Composition D above were prepared and stored in aluminum foil tubes at 25° C. and 60% relative humidity. Percent MCHCl in each sample was measured by HPLC over a period of 6 months.

HPLC was performed using the following parameters:

TABLE 12
HPLC Parameters

| Column: | Alltech Apollo C18, 5 μm, 4.6 × 250 mm or | | |
|---|---|---|---|
| | equivalent | | |
| Flow rate: | 1.2 mL/minute | | |
| Run time: | 22 minutes | | |
| Column temperature: | 45° C. | | |
| Autosampler | Ambient | | |
| temperature: | | | |
| Injection volume: | 5 μL | | |
| Detector: | Ultraviolet detector at 276 nm | | |
| Mobile phase: | A: 5 mM H₃PO₄ in H₂O, pH adjusted to 3 with | | |
| | triethylamine | | |
| | B: Acetonitrile | | |
| | Time | | |
| | (minutes) | % A | % B |
| Gradient: | 0 | 70 | 30 |
| | 2 | 70 | 30 |
| | 8 | 10 | 90 |
| | 12 | 0 | 100 |
| | 12.5 | 0 | 100 |
| | 14 | 70 | 30 |
| | 22 | 70 | 30 |

Samples were prepared for HPLC analysis as follows: A nitrogen mustard sample (25 mg) was removed from the aluminum foil tube, combined with a 150 mM NaCl solution (0.5 mL), and mixed. The resulting solution was then combined with a 10% solution of DDTC in sodium hydroxide (100 μL), mixed, and then incubated at 37° C. for 30 minutes. The resulting solution was then allowed to cool to room temperature and extracted three times with 1.0 mL ethyl acetate. The combined organic extracts were then dried over anhydrous K₂CO₃, the K₂CO₃ was filtered, and the filtrate was evaporated to dryness to form a residue. The residue was then reconstituted with 1.0 mL of acetonitrile and filtered through a 0.2 μm nylon syringe filter.

The results are summarized in Tables 13, 14, and 15 below.

TABLE 13
Results for 0.02% Ointment Batch 1

| | Average % of |
|---|---|
| Time | Label Concentration |
| (mos.) | (by HPLC assay) |
| 0 | 99.6% |
| 1 | 99.2% |
| 2 | 95.2% |
| 3 | 86.1% |
| 4 | 76.2% |
| 5 | 70.5% |
| 6 | 65.6% |
| 7 | 61.2% |

*Averages were taken from 3 to 6 samples.

As illustrated by the data, at least about 85% of the MCHCl is present in 0.02% ointment batch 1 after storage for up to about 3 months; and at least about 65% of the MCHCl is present in 0.02% ointment batch 1 after storage for up to about 6 months.

TABLE 14
Results for 0.02% Ointment Batch 2

| | Average % of |
|---|---|
| Time | Label Concentration |
| (mos.) | (by HPLC assay) |
| 0 | 102.8% |
| 2 | 89.7% |

*Averages were taken from 3 to 6 samples.

As illustrated by the data, at least about 85% of the MCHCl is present in 0.02% ointment batch 2 after storage for up to about 2 months.

TABLE 15
Results for 0.02% Ointment Batch 3

| | Average % of |
|---|---|
| Time | Label Concentration |
| (mos.) | (by HPLC assay) |
| 0 | 102.9% |
| 2 | 89.4% |

*Averages were taken from 3 to 6 samples.

As illustrated by the data, at least about 85% of the MCHCl is present in 0.02% ointment batch 3 after storage for up to about 2 months.

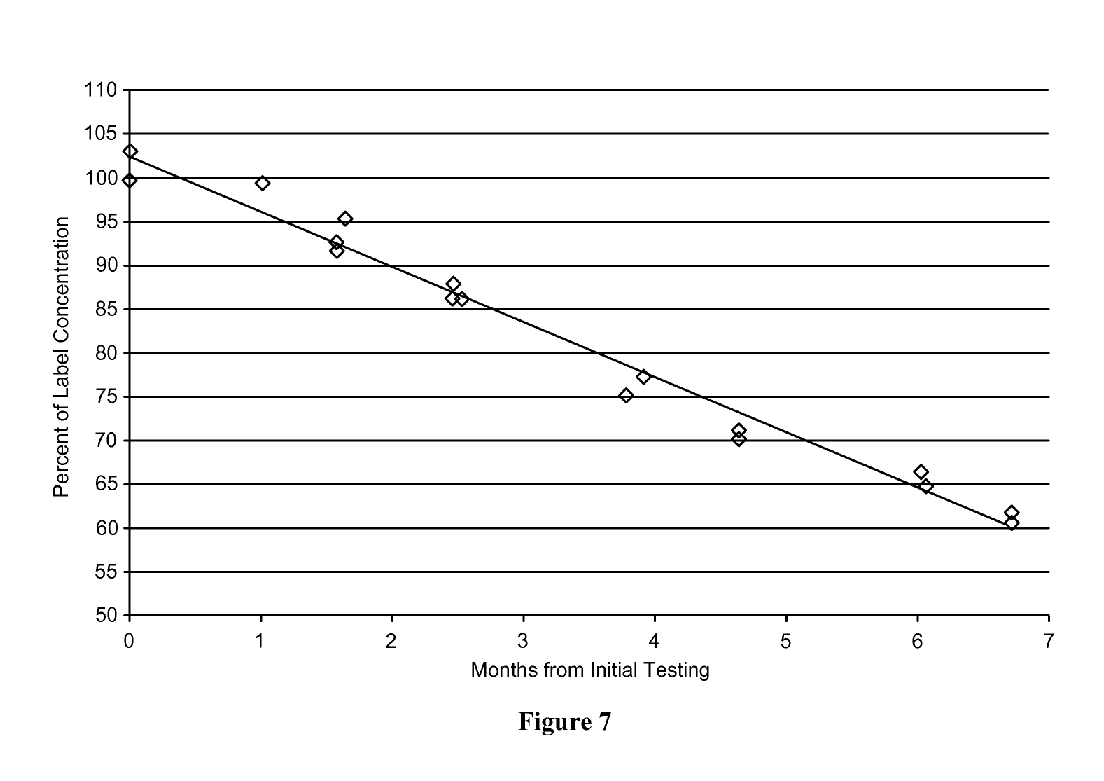

The results for 0.02% ointment batches 1, 2, and 3 stored at 25° C. and 60% relative humidity are also illustrated in FIG. 7.

### Example 9

### Stability of 0.02% w/w Nitrogen Mustard Ointment Batches after Storage Under Refrigeration

Several batches of ointment having 0.02% w/w mechlorethamine HCl were prepared according to Composition D above and stored in aluminum foil tubes under refrigeration (2° C. to 8° C.). Percent MCHCl in each batch was measured by HPLC over time using the HPLC method described in Example 8. The results are summarized in Tables 16 to 19 below.

TABLE 16
Results for 0.02% Ointment Batch 1

| Time | Average % of Label Concentration (by |
|---|---|
| (mos.) | HPLC assay) |
| 0 | 101.9% |
| 6 | 102.2% |
| 12 | 97.0% |
| 25 | 97.5% |
| 31 | 95.4% |

*Averages were taken from 3 samples.

As illustrated by the data, at least about 95% of the MCHCl is present in 0.02% ointment batch 1 after storage for up to about 12 months, 25 months, or 31 months.

TABLE 17
Results for 0.02% Ointment Batch 2

| Time | Average % of Label Concentration (by |
|---|---|
| (mos.) | HPLC assay) |
| 0 | 103.7% |
| 4 | 103.1% |
| 7 | 107.0% |
| 9 | 104.3% |
| 12 | 94.3% |
| 19 | 98.7% |
| 24 | 97.7% |

*Averages were taken from 3 to 6 samples.

As illustrated by the data, at least about 90% of the MCHCl is present in 0.02% ointment batch 2 after storage for up to about 12 months, 19 months, or 24 months.

TABLE 18
Results for 0.02% Ointment Batch 3

| Time | Average % of Label Concentration (by |
|---|---|
| (mos.) | HPLC assay) |
| 0 | 107.5% |
| 3 | 99.5% |
| 7 | 101.3% |
| 9 | 97.6% |
| 12 | 95.8% |
| 18 | 97.7% |

*Averages were taken from 3 samples.

As illustrated by the data, at least about 95% of the MCHCl is present in 0.02% ointment batch 3 after storage for up to about 7 months, 12 months, or 18 months.

TABLE 19
Results for 0.02% Ointment Batch 4

| Time | Average % of Label Concentration (by |
|---|---|
| (mos.) | HPLC assay) |
| 0 | 100.1% |
| 5 | 102.9% |
| 6 | 101.9% |

*Averages were taken from 3 samples.

As illustrated by the data, at least about 95% of the MCHCl is present in 0.02% ointment batch 4 after storage for up to about 6 months.

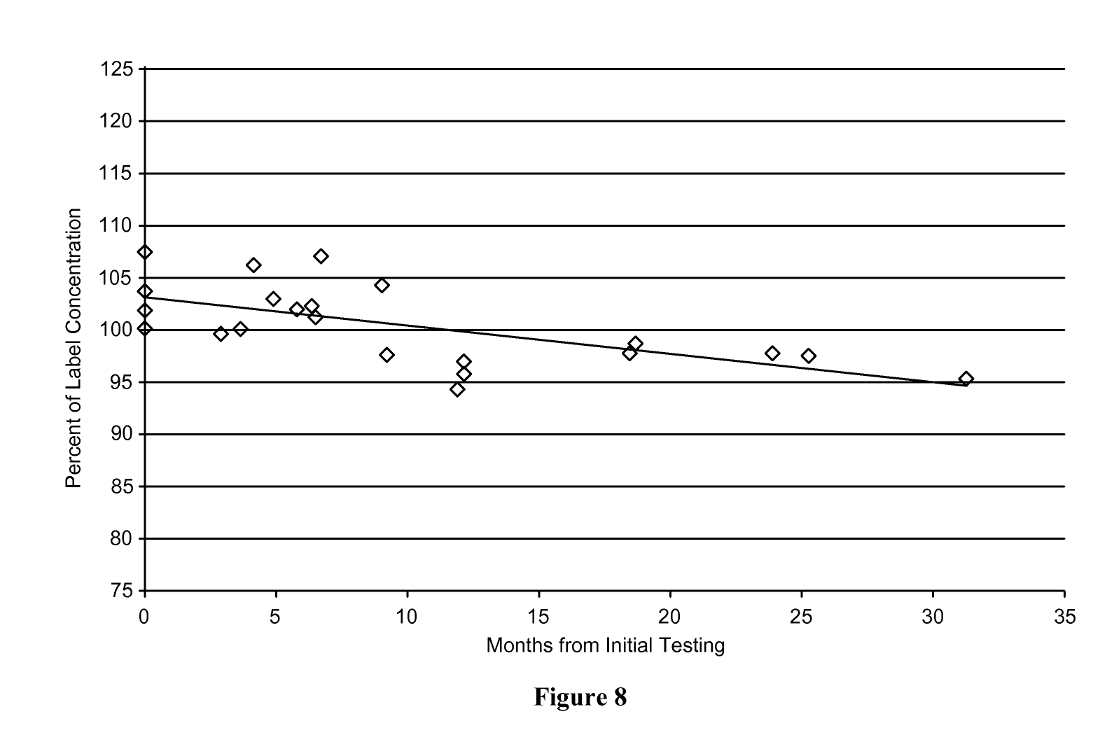

The results for 0.02% ointment batches 1, 2, 3, and 4 stored at 2-8° C. are also illustrated in FIG. 8.

### Example 10

### Stability of 0.04% w/w Nitrogen Mustard Ointment Samples after Storage Under Refrigeration

Several batches of ointment having 0.04% w/w mechlorethamine HCl were prepared according to Composition D above and stored in aluminum foil tubes under refrigeration (2° C. to 8° C.). Percent MCHCl in each batch was measured by HPLC over time using the HPLC method described in Example 8. The results are summarized in Tables 20 to 22 below.

TABLE 20
Results for 0.04% Ointment Batch 1

| Time | Average % of Label Concentration (by |
|---|---|
| (mos.) | HPLC assay) |
| 0 | 116.4% |
| 2 | 93.7% |
| 3 | 86.4% |
| 5 | 105.2% |
| 6 | 103.3% |
| 7 | 107.7% |
| 8 | 106.7% |
| 9 | 104.0% |
| 10 | 94.9% |
| 12 | 102.8% |
| 15 | 98.8% |
| 18 | 98.5% |
| 24 | 92.9% |

*Averages were taken from 3 to 9 samples.

As illustrated by the data, at least about 90% of the MCHCl is present in 0.04% ointment batch 1 after storage for up to about 6 months, 12 months, or 24 months.

TABLE 21
Results for 0.04% Ointment Batch 2

| Time | Average % of Label Concentration (by |
|---|---|
| (mos.) | HPLC assay) |
| 0 | 105.5% |
| 2 | 110.9% |
| 5 | 98.7% |
| 8 | 97.0% |
| 11 | 97.2% |
| 14 | 98.7% |
| 17 | 97.0% |
| 23 | 95.5% |

*Averages were taken from 3 to 6 samples.

As illustrated by the data, at least about 95% of the MCHCl is present in 0.04% ointment batch 2 after storage for up to about 5 months, 11 months, 17 months, or 23 months.

TABLE 22
Results for 0.04% Ointment Batch 3

| Time | Average % of Label Concentration (by |
|---|---|
| (mos.) | HPLC assay) |
| 0 | 101.6% |
| 3 | 99.0% |
| 6 | 97.9% |
| 9 | 99.9% |
| 12 | 98.0% |
| 15 | 98.1% |
| 18 | 98.9% |

*Averages were taken from 3 samples.

As illustrated by the data, at least about 98% of the MCHCl is present in 0.04% ointment batch 3 after storage for up to about 6 months, 9 months, 12 months, 15 months, or 18 months.

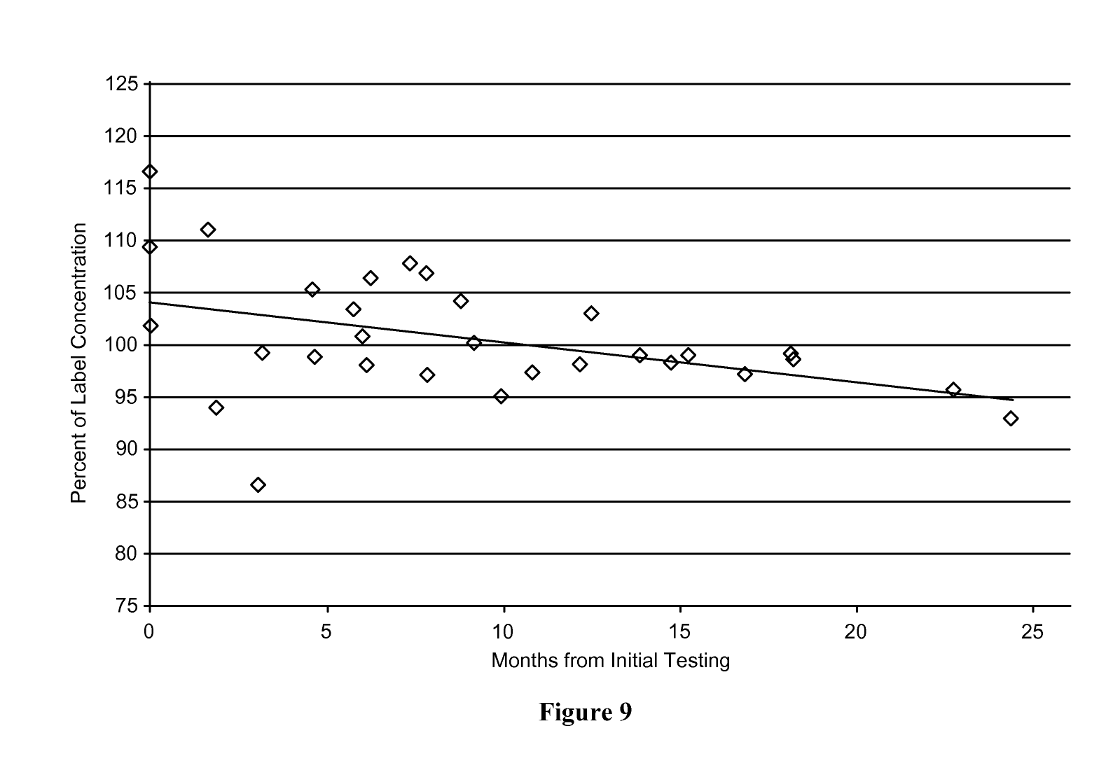

The results for 0.02% ointment batches 1, 2, and 3 stored at 2-8° C. are also illustrated in FIG. 9.

### Example 11

### Identifying and Quantifying Degradation Products in MCHCl Ointments

Three batches of ointment having 0.02% w/w mechlorethamine HCl were prepared according to Composition D above and stored in screw cap ointment tubes under refrigeration (2° C. to 8° C.) for various lengths of time. Batch 1 was stored for 6 months, Batch 2 was stored for 13 months, and Batch 3 was stored for 22 months. The presence of the nitrogen mustard degradation products in each batch was determined by HPLC-MS, and compared to that of placebo (ointment without mechlorethamine HCl).

HPLC-MS was performed using the following parameters:

TABLE 23
HPLC-MS Parameters

| Device: | Agilent HP1100 HPLC system equipped with diode | | |
|---|---|---|---|
| | array detector | | |
| | Micromass QTOF-API US mass spectromater | | |
| | MassLynx 4.0 with SP 4 | | |
| | Micromass QTOF-Ultima mass spectromater | | |
| | MassLynx 4.0 with SP 4 | | |
| Column: | Water Symmetry ® C18 column, 3.5μ, 100 \acute{Å}, | | |
| | 150 × 2.1 mm | | |
| Column Temp.: | 25° C. | | |
| Flow rate: | 0.2 mL/minute | | |
| Run time: | 60 minutes | | |
| Cone voltage: | 35 V, 75 V | | |
| Mobile phase: | A: 0.1% formic acid in water | | |
| | B: 0.1% formic acid in acetonitrile:water (95:5) | | |
| | Time (minutes) | % A | % B |
| Gradient: | 0 | 99% | 1% |
| | 3.10 | 1% | 99% |
| | 22 | 1% | 99% |
| | 41 | 99% | 1% |
| | 50 | 99% | 1% |
| | 60 | 99% | 1% |

MCHCl was found to have a m/z value of 156.0347. The calculated m/z values for several proposed nitrogen mustard degradation products that may be formed from nucleophiles present in Composition D or in the environment (e.g., water) are shown in Table 24 below.

TABLE 24

| | | Calculated m/z |
|---|---|---|
| Nucleophile | Proposed Nitrogen Mustard Degradation Product | value |
| Water | 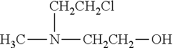 | 138.0697 |
| | (DP-1) | |
| | 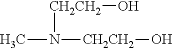 | 119.6243 |
| | (DP-2) | |
| Edetate disodium | 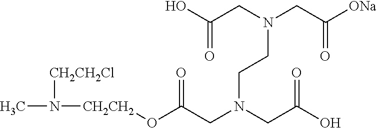 | 457.8 |
| | (DP-3) | |
| | 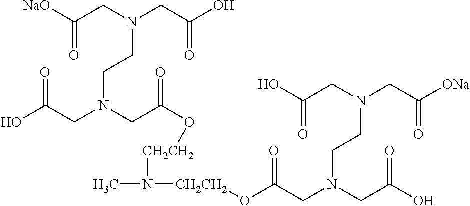 | 758.55 |
| | (DP-4) | |
| Glycerin | 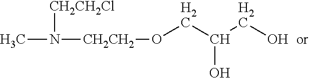 | 213.69 |
| | 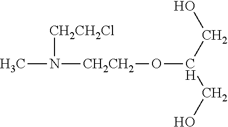 | |
| | (DP-5) | |
| | 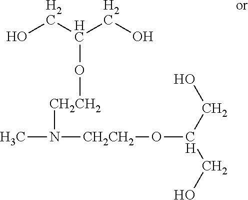 | 270.33 |
| | 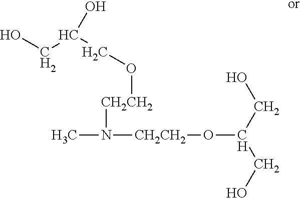 | |
| | 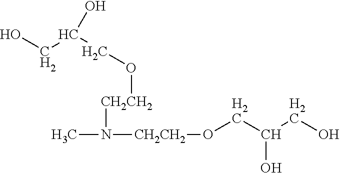 | |
| | (DP-6) | |
| Isopropyl alcohol | 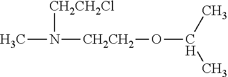 | 181.7 |
| | (DP-7) | |
| | 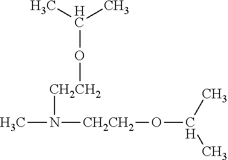 | 206.35 |
| | (DP-8) | |
| 2-(2-ethoxy ethoxy)ethanol | 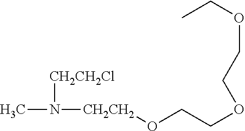 | 255.77 |
| | (DP-9) | |
| | 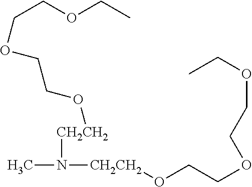 | 354.49 |
| | (DP-10) | |
| Butylated hydroxytoluene | 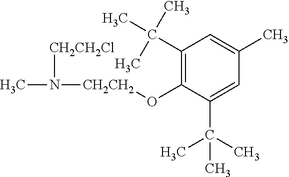 | 341.95 |
| | (DP-11) | |
| | 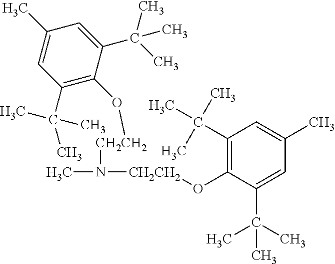 | 526.85 |
| | (DP-12) | |
| Menthol | 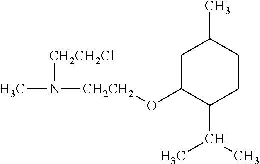 | 277.87 |
| | (DP-13) | |
| | 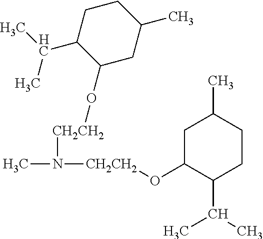 | 398.69 |
| | (DP-14) | |
| Propylene glycol | 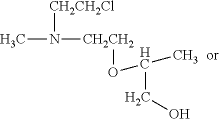 | 196.1104 |
| | (DP-15) | |
| Lactic acid | 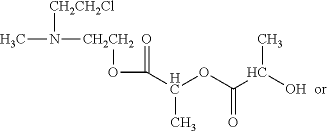 | 282.1128 |
| | 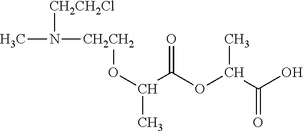 | |
| | (DP-16) | |

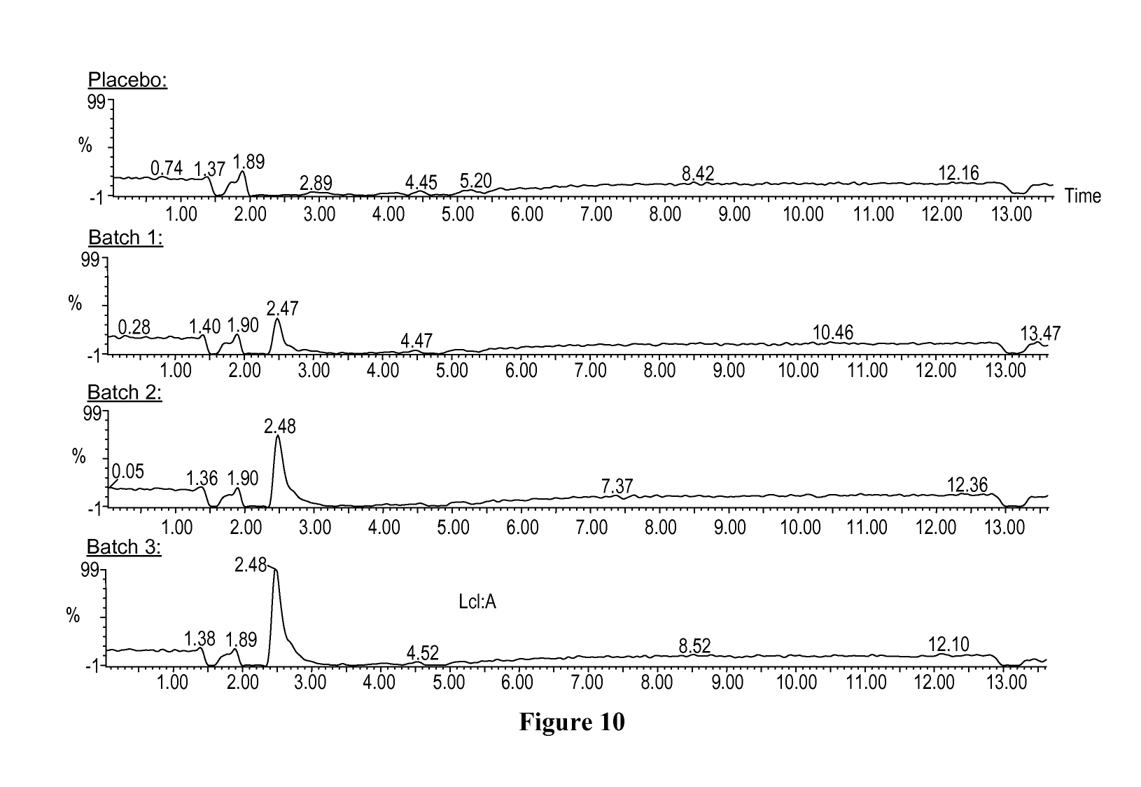

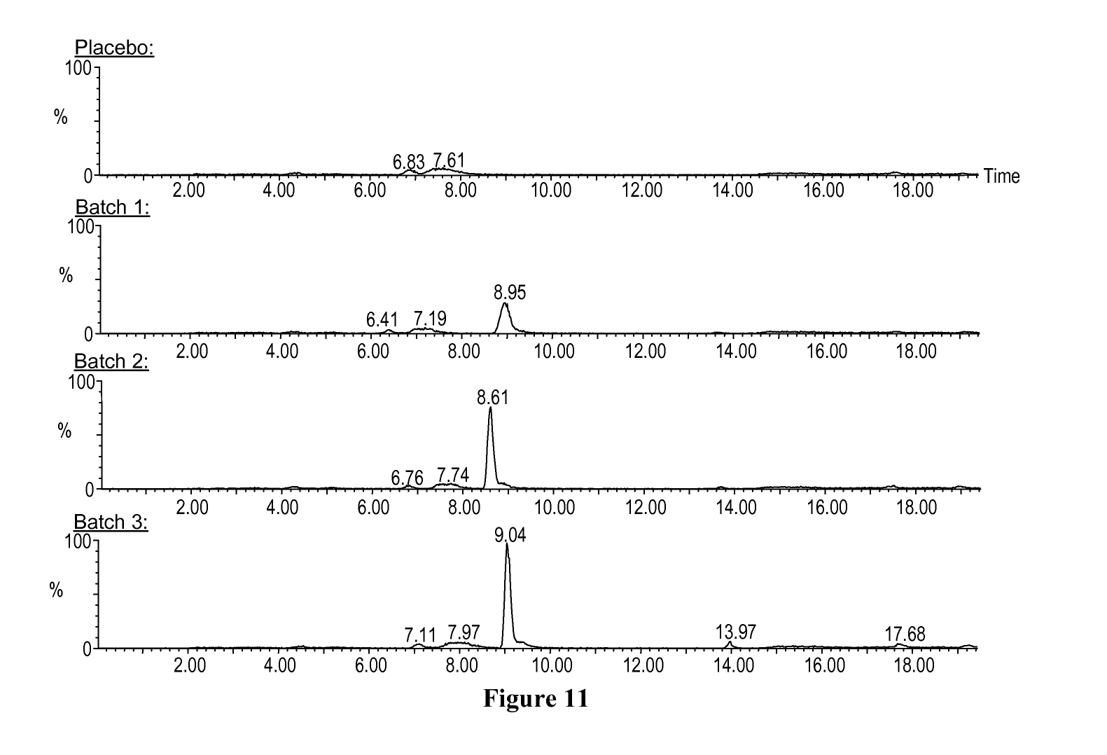

Surprisingly, none of degradation products (DP-1) to (DP-14) were detected in any of Batches 1, 2, or 3. Degradation products (DP-15) and (DP-16), however, were detected in all three batches, as illustrated by FIGS. 10 and 11, which represent the extracted ion currents for m/z 196 (i.e., (DP-15)) and 282 (i.e., (DP-16)), respectively.

### Example 12

### Effect of Temperature on Nitrogen Mustard Degradation Products

Four batches of ointment having 0.02% w/w mechlorethamine HCl were prepared according to Composition D above and stored for 24 hours at various temperatures. Batch 1 was stored at 2° C. to 8° C., Batch 2 was stored at 15° C. to 20° C., and Batch 3 was stored at 50° C. Batch 4 was stored at 15° C. to 20° C. and was spiked with 1% water. The presence of the nitrogen mustard degradation products in each batch was determined by LC-MS. The results are summarized in Table 25 below.

TABLE 25

| | Batch 1 | Batch 2 | | Batch 4 |
|---|---|---|---|---|
| Degradation | (2° C. to | (15° C. to | Batch 3 | (15° C. to 20° C. + |
| Product | 8° C.) | 20° C.) | (50° C.) | 1% water) |
| (DP-15) | 1% | 1% | 9% | 1% |
| (DP-16) | 0% | 0% | 10% | 0% |
| Half-mustard | 0% | 0% | 1% | 0% |
| Total degradation | 1% | 1% | 20% | 1% |
| product | | | | |
| LCMS (assay) | 98% | 97% | 92% | 96% |

As illustrated by the data in Table 25, the amount of nitrogen mustard degradation product formed did not change significantly upon storage between 2° C. and 20° C., but increased when the storage temperature was raised to 50° C. Further, the addition of water did not affect the amount of nitrogen mustard degradation product formed.

### Example 13

### Administration of MCHCl to Patients with Mycosis Fungoides

255 human patients diagnosed with stage I or IIA (cutaneous only mycosis fungoides confirmed by a skin biopsy were topically administered a 0.02% composition of mechlorethamine hydrochloride once daily. A thin layer of the composition was applied to the affected area of skin (i.e., lesions only or total skin surface depending on the severity of the mycosis fungoides) at approximately the same time each day for up to 12 months.

Patients were split into two treatment arms. One treatment arm received 0.02% Composition D (“NM PG 0.02%”) and the other treatment arm received 0.02% mechlorethamine hydrochloride in aquaphor (“NM AP 0.02%”).

The disposition of patients by analysis population is summarized in Table 26 below.

TABLE 26
Disposition of Patients by Analysis Population

| | NM PG 0.02% | | | NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| Population | (N = 76) | (N = 54) | (N = 130) | (N = 65) | (N = 65) | (N = 130) | (N = 260) |
| Intent-to-Treat^{a} | 76 | 54 | 130 | 65 | 65 | 130 | 260 |
| Safety-Evaluable^{b} | 75 | 53 | 128 | 63 | 64 | 127 | 255 |
| Technically-Evaluable^{c} | 62 | 46 | 108 | 52 | 62 | 114 | 222 |
| Efficacy-Evaluable^{d} | 49 | 41 | 90 | 41 | 54 | 95 | 185 |
| Completer^{e} | 45 | 36 | 81 | 36 | 50 | 86 | 167 |
| Modified Intent-to-Treat^{b} | 75 | 53 | 128 | 63 | 64 | 127 | 255 |

^{a}Subjects who were randomized comprise the intent-to-treat population.
^{b}All subjects who were treated with at least one dose of study medication comprise the safety-evaluable and modified intent-to-treat populations.
^{c}Subjects who were treated for at least 2 months are included in the technically evaluable population.
^{d}All subjects who were in the intent-to-treat population and met the identified criteria were in the efficacy-evaluable population.
^{e}Subjects who completed treatment for the 12 month study period are included in the completer population.

Inclusion/Exclusion Criteria

Inclusion criteria for patients were as follows:
A diagnosis of stage I or IIA (cutaneous only) mycosis fungoides confirmed by a skin biopsy.
Patients must not have used steroids for at least four (4) weeks before the diagnostic skin biopsy.
Patients must have been treated previously with topical therapies for MF including PUVA, UVB, topical steroids, but not NM (within the last two years), or topical carmustine (BCNU).
Patients must be free of serious concurrent illness.
Males and females of childbearing potential must be using an effective means of contraception. Females must have a negative pregnancy test.

Exclusion criteria for patients were as follows:
Newly diagnosed MF with no prior therapy.
A prior history of treatment with topical NM (within the last two years) or topical carmustine.
Use of topical or systemic therapies, including corticosteroids, for MF within four (4) weeks of entry in the study.
Patients with a diagnosis of stage IIB-IV MF
Patients who had a history of a higher T score than T2 or a higher N score than N1.
Patients who had radiation therapy within 1 year of study start.
Pregnant or nursing females, or males and females of childbearing potential, not using an effective means of contraception.
Serious known concurrent medical illness or infection, which could potentially present a safety risk and/or prevent compliance with the requirements of the treatment program.

### Patient Characteristics at Baseline (Before Treatment)

The characteristics of the patients are summarized in Tables 27 to 31 below.

TABLE 27
Demographic and Baseline Mycosis Fungoides Stage for the Intent-to-Treat Patients

| | NM PG 0.02% | | NM AP 0.02% | | | |
|---|---|---|---|---|---|---|
| | (N = 130) | | (N = 130) | | | |
| | MF | MF | MF | MF | | |
| | Stage | Stage | Stage | Stage | All | |
| | IA | IB, IIA | IA | IB, IIA | Subjects | |
| Characteristic | (N = 76) | (N = 54) | (N = 65) | (N = 65) | (N = 260) | p-Value^{a} |
| Gender, n (%) | | | | | | >0.999 |
| Male | 40 (52.6) | 37 (68.5) | 43 (66.2) | 34 (52.3) | 154 (59.2) | |
| Female | 36 (47.4) | 17 (31.5) | 22 (33.8) | 31 (47.7) | 106 (40.8) | |
| Race, n (%) | | | | | | 0.840 |
| Caucasian | 61 (80.3) | 36 (66.7) | 57 (87.7) | 39 (60.0) | 193 (74.2) | |
| African-American | 8 (10.5) | 8 (14.8) | 4 (6.2) | 15 (23.1) | 35 (13.5) | |
| Other | 7 (9.2) | 10 (18.5) | 4 (6.2) | 11 (16.9) | 32 (12.3) | |
| Age, y | | | | | | 0.271 |
| N | 76 | 54 | 65 | 65 | 260 | |
| Mean (SD) | 54.1 (13.80) | 55.6 (14.84) | 57.7 (14.05) | 55.7 (14.67) | 55.7 (14.28) | |
| Median | 55.0 | 59.0 | 62.0 | 57.0 | 58.0 | |
| Interquartile Range | (44.0, 64.0) | (44.0, 67.0) | (49.0, 67.0) | (49.0, 65.0) | (46.0, 66.0) | |
| (Q1, Q3) | | | | | | |
| Range (min, max) | (25, 83) | (24, 78) | (23, 82) | (11, 88) | (11, 88) | |
| Age Group, n (%) | | | | | | 0.712 |
| <18 | 0 (0.0) | 0 (0.0) | 0 (0.0) | 1 (1.5) | 1 (0.4) | |
| 18 to 64 | 58 (76.3) | 35 (64.8) | 39 (60.0) | 47 (72.3) | 179 (68.8) | |
| 65 to 74 | 13 (17.1) | 16 (29.6) | 22 (33.8) | 11 (16.9) | 62 (23.8) | |
| ≧75 | 5 (6.6) | 3 (5.6) | 4 (6.2) | 6 (9.2) | 18 (6.9) | |

^{a}Comparisons made between treatment groups. Gender, race, and age group were analyzed with Fisher's exact tests. Age was analyzed with Student's ‘t’ test.

TABLE 28
Mycosis Fungoides History and Previous Treatments for the Intent-to-Treat Patients

| | NM PG 0.02% | | | NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| MF History | (N = 76) | (N = 54) | (N = 130) | (N = 65) | (N = 65) | (N = 130) | (N = 260) |
| Time Since Diagnosed | | | | | | | |
| <6 months | 28 (36.8) | 19 (35.2) | 47 (36.2) | 18 (27.7) | 27 (41.5) | 45 (34.6) | 92 (35.4) |
| 6 months to 1 year | 11 (14.5) | 7 (13.0) | 18 (13.8) | 11 (16.9) | 11 (16.9) | 22 (16.9) | 40 (15.4) |
| 1 to 2 years | 6 (7.9) | 8 (14.8) | 14 (10.8) | 6 (9.2) | 7 (10.8) | 13 (10.0) | 27 (10.4) |
| ≧2 years | 31 (40.8) | 20 (37.0) | 51 (39.2) | 30 (46.2) | 20 (30.8) | 50 (38.5) | 101 (38.8) |
| Previous Treatment/Therapy | 76 (100.0) | 54 (100.0) | 130 (100.0) | 65 (100.0) | 65 (100.0) | 130 (100.0) | 260 (100.0) |
| for MF | | | | | | | |
| Corticosteroids | 63 (82.9) | 49 (90.7) | 112 (86.2) | 52 (80.0) | 61 (93.8) | 113 (86.9) | 225 (86.5) |
| Interferons | 1 (1.3) | 2 (3.7) | 3 (2.3) | 2 (3.1) | 3 (4.6) | 5 (3.8) | 8 (3.1) |
| Methotrexate | 1 (1.3) | 2 (3.7) | 3 (2.3) | 2 (3.1) | 1 (1.5) | 3 (2.3) | 6 (2.3) |
| Phototherapy | 26 (34.2) | 24 (44.4) | 50 (38.5) | 31 (47.7) | 22 (33.8) | 53 (40.8) | 103 (39.6) |
| Radiation | 0 (0.0) | 3 (5.6) | 3 (2.3) | 1 (1.5) | 1 (1.5) | 2 (1.5) | 5 (1.9) |
| Targretin | 13 (17.1) | 10 (18.5) | 23 (17.7) | 12 (18.5) | 11 (16.9) | 23 (17.7) | 46 (17.7) |
| Topical Nitrogen Mustards | 12 (15.8) | 4 (7.4) | 16 (12.3) | 9 (13.8) | 4 (6.2) | 13 (10.0) | 29 (11.2) |
| Other | 9 (11.8) | 5 (9.3) | 14 (10.8) | 18 (27.7) | 16 (24.6) | 34 (26.2) | 48 (18.5) |

TABLE 29
Baseline CAILS By Baseline Mycosis Fungoides Stage for the Intent-to-Treat
Patients

| | NM PG 0.02% | | | NM AP 0.02% | | | |
|---|---|---|---|---|---|---|---|
| | MF | MF | | MF | MF | | |
| | Stage | Stage | | Stage | Stage | | All |
| Baseline CAILS | IA | IB, IIA | Total | IA | IB, IIA | Total | Subjects |
| scores | (N = 76) | (N = 54) | (N = 130) | (N = 65) | (N = 65) | (N = 130) | (N = 260) |
| CAILS Score | | | | | | | |
| N | 75 | 54 | 129 | 63 | 64 | 127 | 256 |
| Mean (SD) | 33.9 (17.88) | 42.1 (16.04) | 37.3 (17.54) | 31.2 (14.40) | 43.5 (18.35) | 37.4 (17.56) | 37.4 (17.52) |
| Median | 31.0 | 39.0 | 34.0 | 27.0 | 42.0 | 34.0 | 34.0 |
| Interquartile Range | (19.0, 46.0) | (31.0, 54.0) | (24.0, 49.0) | (23.0, 39.0) | (31.0, 55.0) | (25.0, 46.0) | (24.5, 47.5) |
| (Q1, Q3) | | | | | | | |
| Range (min, max) | (6, 79) | (2, 74) | (2, 79) | (7, 72) | (6, 87) | (6, 87) | (2, 87) |
| CAILS Score, n (%) | | | | | | | |
| 0 to 25 | 30 (39.5) | 8 (14.8) | 38 (29.2) | 24 (36.9) | 9 (13.8) | 33 (25.4) | 71 (27.3) |
| 26 to 50 | 29 (38.2) | 31 (57.4) | 60 (46.2) | 33 (50.8) | 38 (58.5) | 71 (54.6) | 131 (50.4) |
| 51 to 75 | 15 (19.7) | 15 (27.8) | 30 (23.1) | 6 (9.2) | 11 (16.9) | 17 (13.1) | 47 (18.1) |
| 76 to 100 | 1 (1.3) | 0 (0.0) | 1 (0.8) | 0 (0.0) | 6 (9.2) | 6 (4.6) | 7 (2.7) |
| 101 to 150 | 0 (0.0) | 0 (0.0) | 0 (0.0) | 0 (0.0) | 0 (0.0) | 0 (0.0) | 0 (0.0) |
| 151 to 210 | 0 (0.0) | 0 (0.0) | 0 (0.0) | 0 (0.0) | 0 (0.0) | 0 (0.0) | 0 (0.0) |
| Missing | 1 (1.3) | 0 (0.0) | 1 (0.8) | 2 (3.1) | 1 (1.5) | 3 (2.3) | 4 (1.5) |

TABLE 30
Baseline SWAT Score By Baseline Mycosis Fungoides Stage for the Intent-to-Treat
Patients

| | NM PG 0.02% | | | NM AP 0.02% | | | |
|---|---|---|---|---|---|---|---|
| | MF | MF | | MF | MF | | |
| | Stage | Stage | | Stage | Stage | | All |
| Baseline SWAT | IA | IB, IIA | Total | IA | IB, IIA | Total | Subjects |
| scores | (N = 76) | (N = 54) | (N = 130) | (N = 65) | (N = 65) | (N = 130) | (N = 260) |
| SWAT Score | | | | | | | |
| N | 75 | 54 | 129 | 63 | 64 | 127 | 256 |
| Mean (SD) | 5.8 (4.46) | 26.3 (18.23) | 14.4 (15.87) | 7.1 (7.85) | 31.1 (22.27) | 19.2 (20.58) | 16.8 (18.48) |
| Median | 5.0 | 21.5 | 9.0 | 5.0 | 24.0 | 11.0 | 10.0 |
| Interquartile Range | (3.0, 8.0) | (14.0, 31.0) | (4.0, 20.0) | (3.0, 8.0) | (16.5, 35.5) | (5.0, 25.0) | (4.5, 23.0) |
| (Q1, Q3) | | | | | | | |
| Range (min, max) | (1, 25) | (4, 104) | (1, 104) | (1, 54) | (7, 104) | (1, 104) | (1, 104) |
| SWAT Score, n (%) | | | | | | | |
| 0 to 25 | 75 (98.7) | 32 (59.3) | 107 (82.3) | 61 (93.8) | 36 (55.4) | 97 (74.6) | 204 (78.5) |
| 26 to 50 | 0 (0.0) | 17 (31.5) | 17 (13.1) | 1 (1.5) | 17 (26.2) | 18 (13.8) | 35 (13.5) |
| 51 to 75 | 0 (0.0) | 4 (7.4) | 4 (3.1) | 1 (1.5) | 5 (7.7) | 6 (4.6) | 10 (3.8) |
| 76 to 100 | 0 (0.0) | 0 (0.0) | 0 (0.0) | 0 (0.0) | 5 (7.7) | 5 (3.8) | 5 (1.9) |
| 101 to 150 | 0 (0.0) | 1 (1.9) | 1 (0.8) | 0 (0.0) | 1 (1.5) | 1 (0.8) | 2 (0.8) |
| 151 to 200 | 0 (0.0) | 0 (0.0) | 0 (0.0) | 0 (0.0) | 0 (0.0) | 0 (0.0) | 0 (0.0) |
| 201 to 250 | 0 (0.0) | 0 (0.0) | 0 (0.0) | 0 (0.0) | 0 (0.0) | 0 (0.0) | 0 (0.0) |
| 251 to 300 | 0 (0.0) | 0 (0.0) | 0 (0.0) | 0 (0.0) | 0 (0.0) | 0 (0.0) | 0 (0.0) |
| Missing | 1 (1.3) | 0 (0.0) | 1 (0.8) | 2 (3.1) | 1 (1.5) | 3 (2.3) | 4 (1.5) |

TABLE 31
Baseline % Affected Body Surface Area (“BSA”) by Baseline Mycosis Fungoides
Stage for the Intent-to-Treat Patients

| | NM PG 0.02% | | | 2NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| Baseline % BSA | (N = 76) | (N = 54) | (N = 130) | (N = 65) | (N = 65) | (N = 130) | (N = 260) |
| % BSA | | | | | | | |
| N | 75 | 54 | 129 | 63 | 64 | 127 | 256 |
| Mean (SD) | 4.9 (3.65) | 21.9 (12.06) | 12.1 (11.78) | 5.9 (5.51) | 27.3 (18.18) | 16.6 (17.19) | 14.3 (14.86) |
| Median | 4.0 | 20.0 | 8.0 | 4.0 | 22.5 | 10.0 | 8.0 |
| Interquartile Range | (3.0, 7.0) | (12.0, 28.0) | (3.0, 18.0) | (3.0, 7.0) | (15.5, 34.0) | (4.0, 23.0) | (4.0, 20.5) |
| (Q1, Q3) | | | | | | | |
| Range (min, max) | (1, 25) | (2, 61) | (1, 61) | (1, 33) | (5, 90) | (1, 90) | (1, 90) |
| % BSA, n (%) | | | | | | | |
| 0 to 5 | 47 (61.8) | 2 (3.7) | 49 (37.7) | 39 (60.0) | 2 (3.1) | 41 (31.5) | 90 (34.6) |
| 6 to 10 | 25 (32.9) | 3 (5.6) | 28 (21.5) | 20 (30.8) | 5 (7.7) | 25 (19.2) | 53 (20.4) |
| 11 to 20 | 2 (2.6) | 23 (42.6) | 25 (19.2) | 2 (3.1) | 22 (33.8) | 24 (18.5) | 49 (18.8) |
| 21 to 30 | 1 (1.3) | 15 (27.8) | 16 (12.3) | 1 (1.5) | 14 (21.5) | 15 (11.5) | 31 (11.9) |
| 31 to 50 | 0 (0.0) | 9 (16.7) | 9 (6.9) | 1 (1.5) | 12 (18.5) | 13 (10.0) | 22 (8.5) |
| 51 to 75 | 0 (0.0) | 2 (3.7) | 2 (1.5) | 0 (0.0) | 6 (9.2) | 6 (4.6) | 8 (3.1) |
| 76 to 100 | 0 (0.0) | 0 (0.0) | 0 (0.0) | 0 (0.0) | 3 (4.6) | 3 (2.3) | 3 (1.2) |
| Missing | 1 (1.3) | 0 (0.0) | 1 (0.8) | 2 (3.1) | 1 (1.5) | 3 (2.3) | 4 (1.5) |

### Assessment of Efficacy

Patients were analyzed during a pre-study visit (screening), a baseline (Day 1) visit, and visits at Months 1, 2, 3, 4, 5, 6, 8, 10, and 12.

The primary endpoint to assess efficacy was Composite Assessment of Index Lesion Score (“CAILS”). The CAILS is determined by: (a) assessing a severity score (0=none to 8=severe) for each of the following symptoms for 5 index lesions: erythemia, scaling, plaque elevation, hypo/hyperpigmentation, and surface area; and (b) adding the severity score for each symptom to obtain a CAILS. The maximum CAILS is 40 and the minimum is 0.

Response was determined when the CAILS at the end of treatment was greater than or equal to 50% lower than the CAILS at baseline. Results are summarized in Tables 32 to 35 below.

TABLE 32
Response for Intent-to-Treat Patients (as determined by CAILS)

| | NM PG | NM AP | | |
|---|---|---|---|---|
| | 0.02% | 0.02% | | |
| | (N = 130) | (N = 130) | | 95% Confidence |
| CAILS Response | n (%) | n (%) | Statistic | Limits |
| Response | 76 (58.5) | 62 (47.7) | | |
| Non-Response | 54 (41.5) | 68 (52.3) | | |
| Ratio of Response | | | 1.226 | 0.974^{a} to 1.552 |
| rates | | | | |
| Difference in | | | 10.77 | −1.21^{b} to 22.75 |
| Response rates | | | | |

^{a}This is the lower 95% confidence limit for estimate of ratio (NM PG 0.02%/NM AP 0.02%). This has to be above 0.75 to indicate non-inferiority.
^{b}This is the lower confidence limit of difference in response rates (NM PG 0.02%-NM AP 0.02%).

As shown in Table 32, upon treatment with Composition D, the response rate in a group of human patients is greater than about 60%.

TABLE 33
Response for Efficacy-Evaluable Patients (as determined by CAILS)

| | NM PG | NM AP | | |
|---|---|---|---|---|
| | 0.02% | 0.02% | | |
| | (N = 90) | (N = 95) | | 95% Confidence |
| CAILS Response | n (%) | n (%) | Statistic | Limits |
| Response | 69 (76.7) | 56 (58.9) | | |
| Non-Response | 21 (23.3) | 39 (41.1) | | |
| Ratio of Response | | | 1.301 | 1.065^{a} to 1.609 |
| rates | | | | |
| Difference in | | | 17.72 | 5.53^{b} to 29.91 |
| Response rates | | | | |

^{a}This is the lower 95% confidence limit for estimate of ratio (NM PG 0.02%/NM AP 0.02%). This has to be above 0.75 to indicate non-inferiority.
^{b}This is the lower confidence limit of difference in response rates (NM PG 0.02%-NM AP 0.02%).

TABLE 34
Categorization of Treatment Response or Dropout Reasons by Baseline Mycosis
Fungoides Stage for Intent-to-Treat Patients (as determined by CAILS)

| | NM PG 0.02% | | | NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| | (N = 76) | (N = 54) | (N = 130) | (N = 65) | (N = 65) | (N = 130) | (N = 260) |
| CAILS Outcome Assessment | n (%) | n (%) | n (%) | n (%) | n (%) | n (%) | n (%) |
| Complete Response | 10 (13.2) | 8 (14.8) | 18 (13.8) | 4 (6.2) | 11 (16.9) | 15 (11.5) | 33 (12.7) |
| Partial Response | 35 (46.1) | 23 (42.6) | 58 (44.6) | 22 (33.8) | 25 (38.5) | 47 (36.2) | 105 (40.4) |
| Stable Disease | 26 (34.2) | 16 (29.6) | 42 (32.3) | 34 (52.3) | 27 (41.5) | 61 (46.9) | 103 (39.6) |
| Progressive Disease | 1 (1.3) | 4 (7.4) | 5 (3.8) | 2 (3.1) | 1 (1.5) | 3 (2.3) | 8 (3.1) |
| Unevaluable | 4 (5.3) | 3 (5.6) | 7 (5.4) | 3 (4.6) | 1 (1.5) | 4 (3.1) | 11 (4.2) |
| No Baseline CAILS | 1 (1.3) | 0 (0.0) | 1 (0.8) | 2 (3.1) | 1 (1.5) | 3 (2.3) | 4 (1.5) |
| Assessment | | | | | | | |
| No Post-baseline CAILS | 3 (3.9) | 3 (5.6) | 6 (4.6) | 1 (1.5) | 0 (0.0) | 1 (0.8) | 7 (2.7) |
| Assessment | | | | | | | |

TABLE 35
Categorization of Treatment Response or Dropout Reasons by Baseline Mycosis
Fungoides Stage for Efficacy-Evaluable Patients (as determined by CAILS)

| | NM PG 0.02% | | | NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| | (N = 49) | (N = 41) | (N = 90) | (N = 41) | (N = 54) | (N = 95) | (N = 185) |
| CAILS Outcome Assessment | n (%) | n (%) | n (%) | n (%) | n (%) | n (%) | n (%) |
| Complete Response | 9 (18.4) | 8 (19.5) | 17 (18.9) | 4 (9.8) | 10 (18.5) | 14 (14.7) | 31 (16.8) |
| Partial Response | 30 (61.2) | 22 (53.7) | 52 (57.8) | 19 (46.3) | 23 (42.6) | 42 (44.2) | 94 (50.8) |
| Stable Disease | 10 (20.4) | 9 (22.0) | 19 (21.1) | 18 (43.9) | 21 (38.9) | 39 (41.1) | 58 (31.4) |
| Progressive Disease | 0 (0.0) | 2 (4.9) | 2 (2.2) | 0 (0.0) | 0 (0.0) | 0 (0.0) | 2 (1.1) |

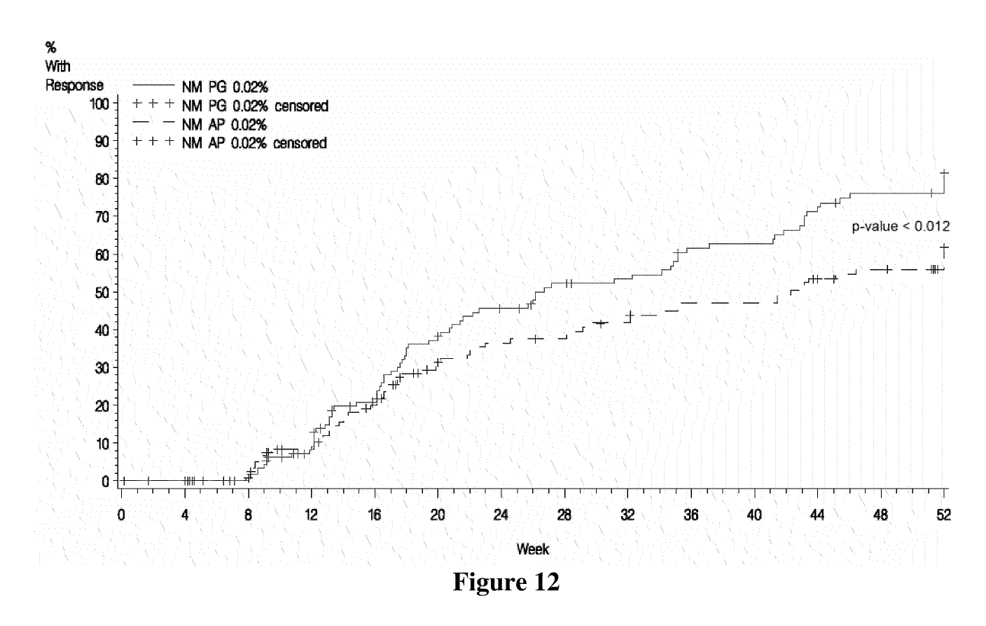

The time to response for intent-to-treat patients (as determined by CAILS) is shown in FIG. 12. As illustrated in FIG. 12, response in 50% of patients is achieved at about 25 weeks for Composition D, and at about 42 weeks for nitrogen mustard in aquaphor.

Secondary efficacy endpoints were Severity Weighted Assessment Tool (“SWAT”) score, improvement in the percent of total body surface area, the time to response, and the time to disease progression. The SWAT score is determined by measuring each lesion as a percentage of total body surface area, and multiplying it by a severity-weighting factor (1=patch, 2=plaque, 3=tumor).

Response was determined when the SWAT score at the end of treatment was greater than or equal to 50% lower than the SWAT score at baseline. Results are summarized in Tables 36 to 39 below.

TABLE 36
Response for Intent-to-Treat Patients (as determined by SWAT score)

| | NM PG | NM AP | | |
|---|---|---|---|---|
| | 0.02% | 0.02% | | |
| | (N = 130) | (N = 130) | | 95% Confidence |
| SWAT Response | n(%) | n(%) | Statistic | Limit |
| Response | 61 (46.9) | 60 (46.2) | | |
| Non-Response | 69 (53.1) | 70 (53.8) | | |
| Difference in | | | 0.77 | −11.36 to 12.90 |
| Response rates^{a} | | | | |

^{a}Difference in response rates (NM PG 0.02%-NM AP 0.02%).
Note:
† Indicates difference in response rates is significant at p < 0.01, from chi-square test.

TABLE 37
Response for Efficacy-Evaluable Patients (as determined by
SWAT score)

| | NM PG | NM AP | | |
|---|---|---|---|---|
| | 0.02% | 0.02% | | |
| | (N = 90) | (N = 95) | | 95% Confidence |
| SWAT Response | n (%) | n (%) | Statistic | Limit |
| Response | 57 (63.3) | 53 (55.8) | | |
| Non-Response | 33 (36.7) | 42 (44.2) | | |
| Difference in | | | 7.54 | −6.35 to 21.44 |
| Response rates^{a} | | | | |

^{a}Difference in response rates (NM PG 0.02%-NM AP 0.02%).
Note:
† Indicates difference in response rates is significant at p < 0.01, from chi-square test.

TABLE 38
Categorization of Treatment Response or Dropout Reasons by Baseline Mycosis
Fungoides Stage for Intent-to-Treat Patients (as determined by SWAT score)

| | NM PG 0.02% | | | NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| SWAT Outcome Assessment | (N = 76) | (N = 54) | (N = 130) | (N = 65) | (N = 65) | (N = 130) | (N = 260) |
| Complete Response | 6 (7.9) | 3 (5.6) | 9 (6.9) | 2 (3.1) | 2 (3.1) | 4 (3.1) | 13 (5.0) |
| Partial Response | 25 (32.9) | 27 (50.0) | 52 (40.0) | 22 (33.8) | 34 (52.3) | 56 (43.1) | 108 (41.5) |
| Stable Disease | 36 (47.4) | 15 (27.8) | 51 (39.2) | 24 (36.9) | 25 (38.5) | 49 (37.7) | 100 (38.5) |
| Progressive Disease | 6 (7.9) | 6 (11.1) | 12 (9.2) | 14 (21.5) | 3 (4.6) | 17 (13.1) | 29 (11.2) |
| Unevaluable | 3 (3.9) | 3 (5.6) | 6 (4.6) | 3 (4.6) | 1 (1.5) | 4 (3.1) | 10 (3.8) |
| No Baseline SWAT | 1 (1.3) | 0 (0.0) | 1 (0.8) | 2 (3.1) | 1 (1.5) | 3 (2.3) | 4 (1.5) |
| Assessment | | | | | | | |
| No Post-baseline SWAT | 2 (2.6) | 3 (5.6) | 5 (3.8) | 1 (1.5) | 0 (0.0) | 1 (0.8) | 6 (2.3) |
| Assessment | | | | | | | |

TABLE 39
Categorization of Treatment Response or Dropout Reasons by Baseline Mycosis
Fungoides Stage for Efficacy-Evaluable Patients (as determined by SWAT score)

| | NM PG 0.02% | | | NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| SWAT Outcome Assessment | (N = 49) | (N = 41) | (N = 90) | (N = 41) | (N = 54) | (N = 95) | (N = 185) |
| Complete Response | 5 (10.2) | 3 (7.3) | 8 (8.9) | 2 (4.9) | 2 (3.7) | 4 (4.2) | 12 (6.5) |
| Partial Response | 23 (46.9) | 26 (63.4) | 49 (54.4) | 18 (43.9) | 31 (57.4) | 49 (51.6) | 98 (53.0) |
| Stable Disease | 17 (34.7) | 9 (22.0) | 26 (28.9) | 14 (34.1) | 19 (35.2) | 33 (34.7) | 59 (31.9) |
| Progressive Disease | 4 (8.2) | 3 (7.3) | 7 (7.8) | 7 (17.1) | 2 (3.7) | 9 (9.5) | 16 (8.6) |
| Unevaluable | 0 (0.0) | 0 (0.0) | 0 (0.0) | 0 (0.0) | 0 (0.0) | 0 (0.0) | 0 (0.0) |
| No Baseline SWAT | 0 (0.0) | 0 (0.0) | 0 (0.0) | 0 (0.0) | 0 (0.0) | 0 (0.0) | 0 (0.0) |
| Assessment | | | | | | | |
| No Post-baseline SWAT | 0 (0.0) | 0 (0.0) | 0 (0.0) | 0 (0.0) | 0 (0.0) | 0 (0.0) | 0 (0.0) |
| Assessment | | | | | | | |

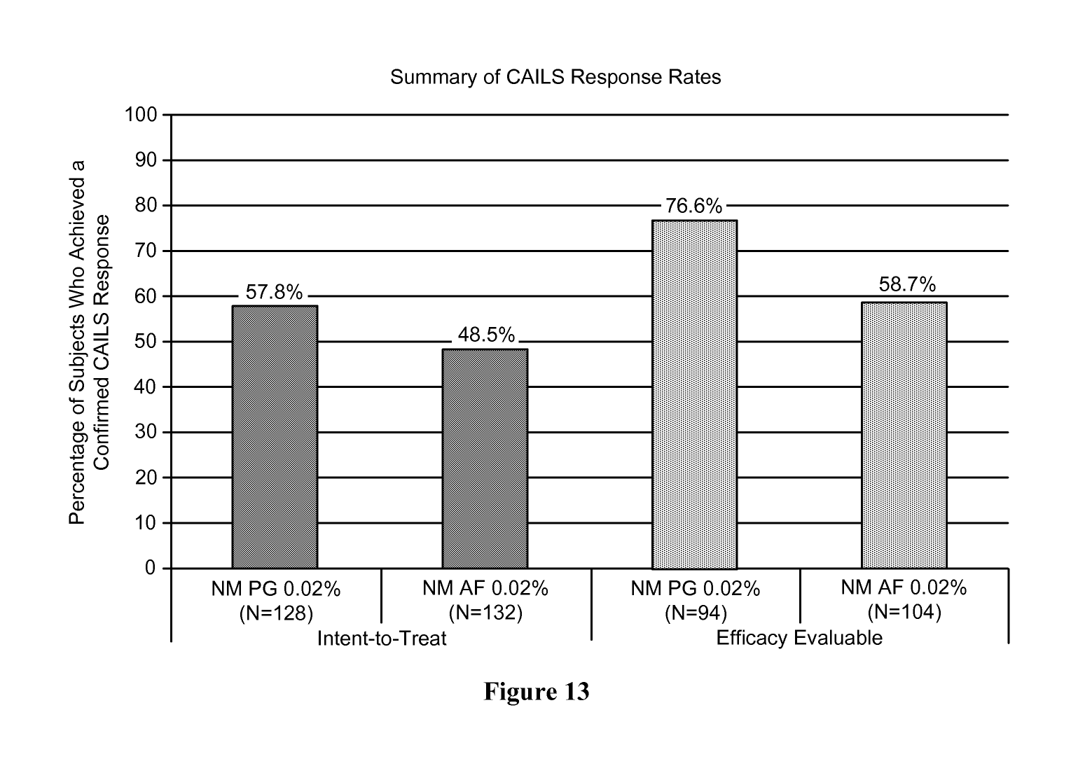

FIG. 13 illustrates a comparison of primary and secondary endpoints for intent-to-treat patients.

### Systemic Absorption

Blood plasma levels of mechlorethamine and a potential mechlorethamine degradation product (N-methyl-(3-chloroethyl)-2-hydroxyethylamine) were measured by HPLC serum assay and LCMS/MS serum assay on day 1 of treatment (at 0, 1, 3, and 6 hours after application of the mechlorethamine composition to the skin), and at 4 weeks of treatment for certain patients.

No mechlorethamine or N-methyl-(β-chloroethyl)-2-hydroxyethylamine was observed in the bloodstream by either assay. For the HPLC assay, the limit of quantitation=45 ng/mL and the sample number was 30. For the LCMS/MS assay, the limit of quantitation was 1.0 ng/mL and the sample number was 5.

### Adverse Events

Adverse events observed are summarized in Tables 40 to 42 below.

TABLE 40
Summary of All Adverse Events

| | NM PG 0.02% | | NM AP 0.02% | | All Subjects | | |
|---|---|---|---|---|---|---|---|
| | (N = 128) | | (N = 127) | | (N = 255) | | |
| Evaluation | n (%) | | n (%) | | n (%) | | p-Value^{a} |
| Subjects with adverse events, n (%) | 108 | (84.4) | 115 | (90.6) | 223 | (87.5) | 0.185 |
| Subjects with serious adverse events, | 14 | (10.9) | 11 | (8.7) | 25 | (9.8) | 0.674 |
| n (%) | | | | | | | |
| Subjects who discontinued due to adverse | 28 | (21.9) | 23 | (18.1) | 51 | (20.0) | 0.532 |
| events^{b}, n (%) | | | | | | | |
| Adverse events^{c}, n | 505 | | 483 | | 988 | | |
| Deaths, n (%) | 0 | (0.0) | 0 | (0.0) | 0 | (0.0) | NC |

^{a}Fisher's exact test.
^{b}Subjects are categorized ‘Discontinued’ if the course of action following an adverse event includes Study Discontinuation.
^{c}Each occurrence of an adverse event is counted, eg, multiple occurrences of the same adverse event within one subject are counted as multiple adverse events.
Note:
NC = Not Calculated.

TABLE 41
Summary of Drug-Related Adverse Events

| | NM PG 0.02% | | NM AP 0.02% | | All Subjects | | |
|---|---|---|---|---|---|---|---|
| | (N = 128) | | (N = 127) | | (N = 255) | | |
| Evaluation | n (%) | | n (%) | | n (%) | | p-Value^{a} |
| Subjects with drug-related^{b} adverse events, | 79 | (61.7) | 64 | (50.4) | 143 | (56.1) | 0.078 |
| n (%) | | | | | | | |
| Subjects with drug-related serious adverse | 0 | (0.0) | 0 | (0.0) | 0 | (0.0) | NC |
| events, n (%) | | | | | | | |
| Subjects who discontinued due to | 27 | (21.1) | 22 | (17.3) | 49 | (19.2) | 0.525 |
| drug-related adverse events^{c}, n (%) | | | | | | | |
| Drug-related adverse events^{d}, n | 206 | | 160 | | 366 | | |
| Drug-related deaths, n (%) | 0 | (0.0) | 0 | (0.0) | 0 | (0.0) | NC |

^{a}Fisher's exact test.
^{b}Drug-related includes adverse events with relation to drug of ‘Yes, related’, ‘Probably related’, ‘Possibly related’ or where such a relationship is not specified.
^{c}Subjects are categorized ‘Discontinued’ if the course of action following a drug-related adverse event includes Study Discontinuation.
^{d}Each occurrence of a drug-related adverse event is counted, eg, multiple occurrences of the same drug-related adverse event within one subject are counted as multiple drug-related adverse events.
Note:
NC = Not Calculated.

TABLE 42
Distribution of Patients Who Permanently Withdrew from the Study Prematurely by
Primary Reason for Withdrawal

| | NM PG 0.02% | | | NM AP 0.02% | | | |
|---|---|---|---|---|---|---|---|
| | MF | MF | | MF | MF | | |
| | Stage | Stage | | Stage | Stage | | All |
| | IA | IB, IIA | Total | IA | IB, IIA | Total | Subjects |
| | (N = 76) | (N = 54) | (N = 130) | (N = 65) | (N = 65) | (N = 130) | (N = 260) |
| Reason for Withdrawal^{a} | n (%) | n (%) | n (%) | n (%) | n (%) | n (%) | n (%) |
| Treatment Limiting | 16 (21.1) | 5 (9.3) | 21 (16.2) | 14 (21.5) | 2 (3.1) | 16 (12.3) | 37 (14.2) |
| Toxicity | | | | | | | |
| Other Adverse Event | 2 (2.6) | 3 (5.6) | 5 (3.8) | 5 (7.7) | 1 (1.5) | 6 (4.6) | 11 (4.2) |
| Concurrent Illness | 2 (2.6) | 2 (3.7) | 4 (3.1) | 1 (1.5) | 2 (3.1) | 3 (2.3) | 7 (2.7) |
| Lack of Efficacy | 2 (2.6) | 2 (3.7) | 4 (3.1) | 1 (1.5) | 3 (4.6) | 4 (3.1) | 8 (3.1) |
| Withdrew Consent | 2 (2.6) | 1 (1.9) | 3 (2.3) | 2 (3.1) | 2 (3.1) | 4 (3.1) | 7 (2.7) |
| Subject's Best Interest | 1 (1.3) | 1 (1.9) | 2 (1.5) | 1 (1.5) | 1 (1.5) | 2 (1.5) | 4 (1.5) |
| Non-Compliance | 1 (1.3) | 1 (1.9) | 2 (1.5) | 1 (1.5) | 2 (3.1) | 3 (2.3) | 5 (1.9) |
| Lost to Follow-up | 3 (3.9) | 1 (1.9) | 4 (3.1) | 2 (3.1) | 1 (1.5) | 3 (2.3) | 7 (2.7) |
| Other | 2 (2.6) | 2 (3.7) | 4 (3.1) | 2 (3.1) | 1 (1.5) | 3 (2.3) | 7 (2.7) |
| Total Withdrawn | 31 (40.8) | 18 (33.3) | 49 (37.7) | 29 (44.6) | 15 (23.1) | 44 (33.8) | 93 (35.8) |

^{a}Subjects were counted once, under their primary reason for withdrawal. For subjects with more than one reason given for withdrawal, the primary reason given was taken using the following hierarchy: Treatment limiting toxicity, other adverse event, concurrent illness, lack of efficacy, withdrew consent, subject's best interest, non-compliance, lost to follow-up, and other reasons.

One of the primary adverse events was skin toxicity. 20% of patients withdrew from the study for skin toxicity in the NM PG 0.02% arm and 16.9% of patients withdrew from the study for skin toxicity in the NM AP 0.02% arm. 24 patients in the NM PG 0.02% arm and 20 patients in the NM AP 0.02% arm withdrew for contact dermatitis. 2 patients in each of the NM PG 0.02% and NM AP 0.02% arms withdrew for hypersensitivity.

Another adverse event was skin cancer. 2 patients in the NM PG 0.02% arm and 4 patients in the NM AP 0.02% arm developed basal cell carcinoma. 1 patient in the NM PG 0.02% arm and 2 patients in the NM AP 0.02% arm developed squamous cell carcinoma.

The following are some illustrative embodiments of the invention:

1. A method for treating a skin disorder comprising topically applying to the affected skin a composition comprising an effective amount of an alkylating agent or a pharmaceutically acceptable salt thereof and a pharmaceutically acceptable excipient, wherein the response rate in a group of human patients is greater than about 60% after at least six months of treatment.
2. The method of embodiment 1, wherein the response rate in a group of human patients is greater than about 65%, greater than about 70%, greater than about 75%, greater than about 80%, greater than about 85%, greater than about 90%, or greater than about 95% after at least six months of treatment.
3. A method for treating a skin disorder comprising topically applying to the affected skin a composition comprising an effective amount of an alkylating agent or a pharmaceutically acceptable salt thereof and a pharmaceutically acceptable excipient, wherein the response rate in an intent-to-treat group of human patients is greater than about 50%.
4. A method for treating a skin disorder comprising topically applying to the affected skin a composition comprising an effective amount of an alkylating agent or a pharmaceutically acceptable salt thereof and a pharmaceutically acceptable excipient, wherein the response rate in a group of human patients is greater than about 55% upon two months of treatment.
5. A method for treating a skin disorder comprising topically applying to the affected skin a composition comprising an effective amount of bis-(2-chloroethyl)methylamine or a pharmaceutically acceptable salt thereof and a pharmaceutically acceptable excipient, wherein the time to achieve a response rate of 50% in a group of human patients is about 30 weeks or less.
6. The method of embodiment 5, wherein the time to achieve a response rate of 50% in a group of human patients is about 25, about 20, about 15, about 10, or about 5 weeks or less.
7. A method for treating a skin disorder comprising topically applying to the affected skin a composition comprising an effective amount of bis-(2-chloroethyl)methylamine or a pharmaceutically acceptable salt thereof and a pharmaceutically acceptable excipient, wherein:

the response rate in a group of human patients is greater than the response rate achieved upon application of a reference composition; and

the reference composition consists of the effective amount of bis-(2-chloroethyl)methylamine or pharmaceutically acceptable salt thereof, a hydrophobic carrier, and an organic solvent.

8. The method of embodiment 7, wherein the response rate is at least about 5%, at least about 10%, at least about 15%, at least about 20%, at least about 25%, at least about 30%, at least about 35%, or at least about 40% greater than the response rate achieved upon application of the reference composition.
9. A method for treating a skin disorder comprising topically applying to the affected skin a composition comprising an effective amount of bis-(2-chloroethyl)methylamine or a pharmaceutically acceptable salt thereof and a pharmaceutically acceptable excipient, wherein:

the time to achieve a response rate of 50% in a group of human patients is less than the time to achieve a response rate of 50% upon application of a reference composition; and

the reference composition consists of the effective amount of bis-(2-chloroethyl)methylamine or pharmaceutically acceptable salt thereof, a hydrophobic carrier, and an organic solvent.

10. The method of embodiment 9, wherein the time to achieve a response rate of 50% in a group of human patients is at least about 10%, at least about 15%, at least about 20%, at least about 25%, at least about 30%, at least about 35%, or at least about 40% less than the time to achieve a response rate of 50% upon application of the reference composition.
11. The method of embodiment 9, wherein the time to achieve a response rate of 50% in a group of human patients is at least about 5, at least about 10, at least about 15, or at least about 20 weeks less than the time to achieve a response rate of 50% achieved upon application of the reference composition.
12. The method of any one of embodiments 7 to 11, wherein the hydrophobic carrier is petrolatum.
13. The method of any one of embodiments 7 to 11, wherein the hydrocarbon carrier is a mixture of petrolatum, mineral oil, ceresin, lanolin, panthenol, glycerin, and bisabolol.
14. The method of any one of embodiments 7 to 13, wherein the organic solvent is ethanol or acetone.
15. The method of any one of embodiments 7 to 11, wherein the reference composition consists of the effective amount of bis-(2-chloroethyl)methylamine or pharmaceutically acceptable salt thereof and aquaphor.
16. The method of any one of embodiments 7 to 15, wherein the percent occurrence of an adverse event in a group of human patients upon application of the composition is substantially equal to the percent occurrence of the adverse event upon application of the reference composition.
17. The method of embodiment 16, wherein the adverse event is skin toxicity, such as allergic contact dermatitis, irritant contact dermatitis, or hypersensitivity, or skin cancer.
18. The method of any one of embodiments 1 to 17, wherein the alkylating agent is a nitrogen mustard.
19. The method of embodiment 18, wherein the nitrogen mustard is a compound of the following Structure (VII), (VIII), (IX), (X), (XII), (XIII), (XIV), (XV), (XVI), (XVII), (XVIII), or (XIX):

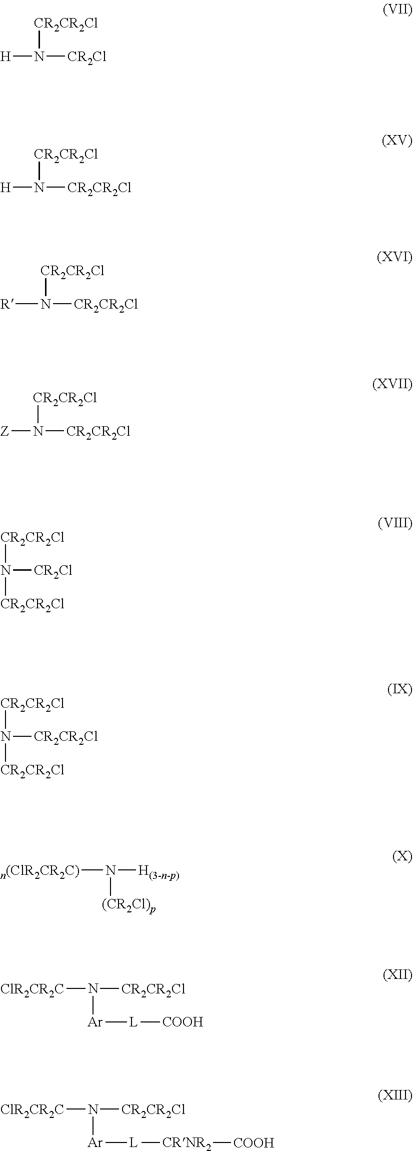

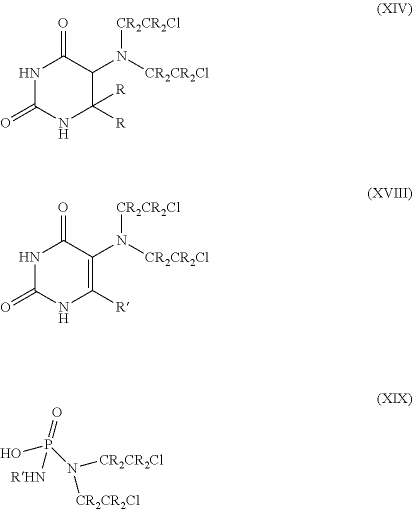

wherein:

each R and R′ is independently selected from the group consisting of H, a linear alkyl group having 1-6 carbon atoms, a branched alkyl group having 2-12 carbon atoms, a cycloalkyl group having 3-17 carbon atoms, a fluorinated linear alkyl group having 2-12 carbon atoms, a fluorinated branched alkyl group having 2-12 carbon atoms, a fluorinated cycloalkyl group having 3-17 carbon atoms, an aryl group, an aralkyl group, an alkaryl group, a cycloalkyl group, a bicycloalkyl group, an alkenyl group, an alkalkenyl group, an alkenylalkyl group, an alkynyl group, an alkalkynyl group, an alkynylalkyl group, a trifluoropropyl group, a cyanopropyl group, an acryloyl group, an arylacryloyl group, an acryloylaryl group, an alkylacyl group, an arylacyl group, an alkylenylacyl group, and an alkynylacyl group, wherein any two R in the same molecule are optionally linked to form a three- to eight-membered cyclic group;

Z is a linear alkyl group having 1-6 carbon atoms;

each L is independently a linking group selected from the group consisting of linear or branched alkylene having 1 to 7 carbon atoms, cycloalkylene having 3 to 17 carbon atoms, alkylcycloalkylene having 4 to 20 carbon atoms, a cycloalkylalkylene having 4 to 20 carbon atoms, an arylene, having 4 to 30 carbon atoms, an alkylarylene, having 4 to 30 carbon atoms, an arylalkylene, having 4 to 30 carbon atoms, and combinations thereof;

each Ar is independently a bifunctional aromatic linking group wherein each Ar is selected from the group consisting of arylene, substituted arylene and heteroarylene;

n is 1, 2, or 3;

p is 0, 1, or 2; and
n+p≦3.
   20. The method of embodiment 18, wherein the nitrogen mustard is a compound of the following Structure (XVII)

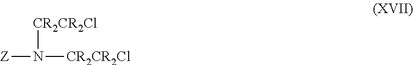

wherein Z is a linear alkyl group having 1-6 carbon atoms and each R is independently hydrogen or a linear alkyl group having 1-6 carbon atoms.
21. The method of embodiment 20, wherein Z is methyl or ethyl.
22. The method of embodiment 18, wherein the nitrogen mustard is bis-(2-chloroethyl)ethylamine, bis-(2-chloroethyl)methylamine, or tris-(2-chloroethyl)amine.
23. The method of embodiment 18, wherein the nitrogen mustard is bis-(2-chloroethyl)methylamine.
24. The method of embodiment 23, wherein the bis-(2-chloroethyl)methylamine is in the form of an acid-addition or base-addition salt.
25. The method of embodiment 24, wherein the acid-addition salt is an HCl, HBr, HI, H₂SO₄, or HNO₃ salt.
26. The method of embodiment 25, wherein the acid-addition salt is an HCl salt.
27. The method of any one of embodiments 23 to 26, wherein the bis-(2-chloroethyl)methylamine or pharmaceutically acceptable salt thereof is present in an amount of about 0.001% to about 2.0% by weight of the composition.
28. The method of any one of embodiments 23 to 26, wherein the bis-(2-chloroethyl)methylamine or pharmaceutically acceptable salt thereof is present in an amount of about 0.01% to about 0.04% by weight of the composition.
29. The method of any one of embodiments 23 to 26, wherein the bis-(2-chloroethyl)methylamine or pharmaceutically acceptable salt thereof is present in an amount of about 0.015% to about 0.030% by weight of the composition.
30. The method of any one of embodiments 23 to 26, wherein the bis-(2-chloroethyl)methylamine or pharmaceutically acceptable salt thereof is present in an amount of about 0.02% by weight of the composition.
31. The method of any one of embodiments 1 to 30, wherein the composition has a viscosity of about 5,000 cps to about 50,000 cps.
32. The method of any one of embodiments 1 to 30, wherein the composition has a viscosity of about 15,000 cps to about 40,000 cps.
33. The method of any one of embodiments 1 to 30, wherein the composition has a viscosity of about 20,000 cps to about 35,000 cps.
34. The method of any one of embodiments 1 to 33, wherein the pharmaceutically acceptable excipient is a diethylene glycol monosubstituted ether.
35. The method of any one of embodiments 1 to 33, wherein the pharmaceutically acceptable excipient is a compound of the formula HOCH₂CH₂OCH₂CH₂OR₇₉, wherein R₇₉ is a linear alkyl group having 1-6 carbon atoms.
36. The method of any one of embodiments 1 to 33, wherein the pharmaceutically acceptable excipient is 2-(2-ethoxyethoxy)ethanol.
37. The method of any one of embodiments 1 to 33, wherein the pharmaceutically acceptable excipient is a polyoxylglyceride.
38. The method of any one of embodiments 1 to 33, wherein the pharmaceutically acceptable excipient is an antioxidant.
39. The composition of embodiment 38, wherein the antioxidant is butylated hydroxytoluene, edetate disodium, or benzyl alcohol.
40. The composition of embodiment 38, wherein the antioxidant is butylated hydroxytoluene and/or edetate disodium.
41. The method of any one of embodiments 1 to 29, wherein the pharmaceutically acceptable excipient is an organic acid.
42. The method of embodiment 41, wherein the organic acid is oxalic acid, p-bromophenylsulfonic acid, carbonic acid, succinic acid, citric acid, benzoic acid, 2-acetoxybenzoic acid, acetic acid, phenylacetic acid, propionic acid, glycolic acid, stearic acid, lactic acid, malic acid, tartaric acid, ascorbic acid, maleic acid, hydroxymaleic acid, glutamic acid, salicylic acid, sulfanilic acid, or fumaric acid.

We claim:

1. A method for treating mycosis fungoides comprising topically applying to the affected skin a composition comprising an effective amount of bis-(2-chloroethyl)methylamine or a pharmaceutically acceptable salt thereof and a pharmaceutically acceptable excipient, wherein the response rate in a group of human patients is greater than about 60% after at least six months of treatment.

2. The method of claim 1, wherein the bis-(2-chloroethyl)methylamine or pharmaceutically acceptable salt thereof is present in an amount of about 0.015% to about 0.030% by weight of the composition.

3. The method of claim 1, wherein the bis-(2-chloroethyl)methylamine or pharmaceutically acceptable salt thereof is present in an amount of about 0.01% to about 0.10% by weight of the composition.

4. A method for treating mycosis fungoides comprising topically applying to the affected skin a composition comprising an effective amount of bis-(2-chloroethyl)methylamine or a pharmaceutically acceptable salt thereof and a pharmaceutically acceptable excipient, wherein the response rate in a group of human patients is greater than about 55% upon two months of treatment.

5. The method of claim 4, wherein the bis-(2-chloroethyl)methylamine or pharmaceutically acceptable salt thereof is present in an amount of about 0.015% to about 0.030% by weight of the composition.

6. The method of claim 4, wherein the bis-(2-chloroethyl)methylamine or pharmaceutically acceptable salt thereof is present in an amount of about 0.01% to about 0.10% by weight of the composition.

7. A method for treating mycosis fungoides comprising topically applying to the affected skin a composition comprising an effective amount of bis-(2-chloroethyl)methylamine or a pharmaceutically acceptable salt thereof and a pharmaceutically acceptable excipient, wherein the response rate in an intent-to-treat group of human patients is greater than about 50%.

8. The method of claim 7, wherein the bis-(2-chloroethyl)methylamine or pharmaceutically acceptable salt thereof is present in an amount of about 0.015% to about 0.030% by weight of the composition.

9. The method of claim 7, wherein the bis-(2-chloroethyl)methylamine or pharmaceutically acceptable salt thereof is present in an amount of about 0.01% to about 0.10% by weight of the composition.

10. A method for treating mycosis fungoides comprising topically applying to the affected skin a composition comprising an effective amount of bis-(2-chloroethyl)methylamine or a pharmaceutically acceptable salt thereof and a pharmaceutically acceptable excipient, wherein the time to achieve a response rate of 50% in a group of human patients is about 40 weeks or less.

11. The method of claim 10, wherein the response rate is about 30 weeks or less.

12. The method of claim 10, wherein the bis-(2-chloroethyl)methylamine or pharmaceutically acceptable salt thereof is present in an amount of about 0.015% to about 0.030% by weight of the composition.

13. The method of claim 10, wherein the bis-(2-chloroethyl)methylamine or pharmaceutically acceptable salt thereof is present in an amount of about 0.01% to about 0.10% by weight of the composition.
